(19) United States
(12) Patent Application Publication
(10) Pub. No.: US 2025/0312793 A1
(43) Pub. Date: Oct. 9, 2025

(54) POROUS STRUCTURE CONFINEMENT FOR CONVECTION SUPPRESSION

(71) Applicant: 10x Genomics, Inc., Pleasanton, CA (US)

(72) Inventors: Lung-Hsin HUNG, Pleasanton, CA (US); Monica NAGENDRAN, Pleasanton, CA (US); Tobias Daniel WHEELER, Pleasanton, CA (US)

(73) Assignee: 10x Genomics, Inc., Pleasanton, CA (US)

(21) Appl. No.: 18/867,759

(22) PCT Filed: May 22, 2023
(86) PCT No.: PCT/US2023/023074

§ 371 Date: Nov. 20, 2024

Related U.S. Application Data
(60) Provisional application No. 63/345,326, filed on May 24, 2022.

(51) Int. Cl. B01L 3/00; C12Q 1/6841

(52) U.S. Cl. CPC B01L 3/508; C12Q 1/6841; B01L 2300/047; B01L 2300/0636; B01L 2300/069; B01L 2300/0822; B01L 2400/0472; B01L 2400/084

(57) ABSTRACT

Provided herein are technologies for reducing fluid convection during processing and analysis of a biological sample. The provided technologies can include the use of a porous insert to limit fluid convection adjacent to a biological sample. In particular, provided technologies can include providing a substrate comprising a capture area, a biological sample comprising a cell disposed on the capture area, a buffer disposed on the biological sample, and a gasket disposed on the substrate, wherein the gasket provides a chamber comprising the lateral dimension of the capture area and includes a height above the capture area and biological sample; and a porous insert positioned in the chamber and in contact with the buffer and/or or the biological sample, wherein the porous insert limits free flow space in the chamber, thereby reducing fluid convection adjacent to the biological sample.

### CROSS-REFERENCE TO RELATED PATENT APPLICATIONS

This application claims the benefit under 35 U.S.C. § 119(e) of U.S. Provisional Patent Application Ser. No. 63/345,326, filed May 24, 2022, the entire contents of which is incorporated herein by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

One challenge associated with the spatial profiling of target analytes is mislocalization, for example mRNA transcript mislocalization, due to convection during processing steps. Accordingly, there is a need in the art for technologies that reduce the impact of convection-induced analyte displacement for improved spatial resolution and profiling of target analytes, such as gene expression profiling, from cells and tissues.

### SUMMARY OF THE INVENTION

In current methodologies used to study the spatial distribution of a target analyte (e.g., target messenger RNA (mRNA)), a tissue is permeabilized by proteinase in a solution, and the tissue associated mRNA transcripts are released and migrate to a proximal location on a spatial array by diffusion and gravity. However, diffusion in addition to convection can create a discrepancy between transcripts' position in tissue and their corresponding bound position on a spatial array (i.e., as on a detection slide). This disclosure provides an engineered insert structure and a method to suppress convection above tissue level and also to limit displacement of an analyte away from its native position in tissue by limiting the free flow space, reducing the number of transcripts bound outside of a region on a capture area immediately adjacent to tissue. An insert structure may be made of porous material, and may include a hydrophilic surface, which allow a permeabilization solution to easily penetrate through the insert structure while keeping the tissue wet during permeabilization.

Provided herein are methods for reducing fluid convection in a chamber. In some embodiments, a method for reducing fluid convection in a chamber comprises providing a substrate comprising a capture area, a biological sample disposed on the capture area, a buffer disposed on the biological sample, and a gasket disposed on the substrate surrounding the capture area, wherein the gasket provides a chamber comprising the lateral dimension of the capture area and includes a height above the capture area and biological sample; and a porous insert positioned in the chamber and in contact with the buffer and/or the biological sample, wherein the porous insert limits free flow space in the chamber, thereby reducing fluid convection above the biological sample. In some embodiments, a method for reducing fluid convection in a chamber comprises providing a substrate comprising a capture area, a biological sample comprising a cell disposed on the capture area, and a gasket disposed on the substrate, wherein the gasket comprises an opening encompassing the lateral dimension of the capture area and includes a height above the capture area and biological sample; and inserting a porous insert in the opening, wherein the porous insert is pre-loaded with a buffer, wherein the porous insert limits free flow space, of the buffer pre-loaded in the porous insert thereby reducing fluid convection above the biological sample.

Provided herein are methods for correlating a location of a biological analyte in a biological sample. In some embodiments, a method for correlating a location of a biological analyte in a biological sample comprises: providing a substrate comprising a capture area and a capture probe attached to the capture area, wherein the capture probe comprises a spatial barcode and a capture domain, a biological sample disposed on the capture area, a buffer disposed on the biological sample, and a gasket disposed on the substrate, wherein the gasket surrounds the capture area and provides a chamber that comprises an opening encompassing the lateral dimension of the capture area and includes a height above the biological sample and buffer; inserting a porous insert in the opening of the chamber wherein the porous insert is in contact with the buffer and/or the biological sample, and wherein the porous insert limits free flow space in the chamber, thereby reducing fluid convection above the biological sample; capturing a biological analyte from the biological sample or an intermediate agent indicative of the presence of the biological analyte in the biological sample using the capture probe; and determining the sequence of the spatial barcode of the capture probe, or a complement thereof, and the sequence of the biological analyte, or a complement thereof, and using the sequences to correlate the location of the biological analyte to its location in the biological sample.

Provided herein are methods for identifying a location of a biological analyte in a biological sample. In some embodiments, a method for identifying a location of a biological analyte in a biological sample comprises: providing a substrate comprising a capture area and a capture probe attached to the capture area, wherein the capture probe comprises a spatial barcode and a capture domain, a biological sample disposed on the capture area, and a gasket disposed on the substrate, wherein the gasket generates a chamber encompassing the lateral dimension of the capture area and a height above the biological sample; inserting a porous insert in the opening, wherein the porous insert is pre-filled with a buffer and is disposed on the biological sample, wherein the porous insert limits free flow space in the chamber, thereby reducing fluid convection above the biological sample; capturing a biological analyte or an intermediate agent indicative of the presence of the biological analyte in the biological sample by the capture probe; and determining the sequence of the spatial barcode of the capture probe, or a complement thereof, and the sequence of the biological analyte from the biological sample, or a complement thereof, and using the sequences to identify the location of the biological analyte in the biological sample.

Provided herein are kits useful for reducing fluid convection around a biological sample. In some embodiments, a kit includes a substrate comprising a capture area for receiving a biological sample; a gasket configured to be disposed on the substrate, wherein when disposed on the substrate the gasket generates a chamber encompassing the lateral dimension of the capture area; and a porous insert configured to be inserted in the chamber, wherein when inserted in the chamber the porous insert defines a height of the gasketed area and limits free flow space in the gasketed area, thereby reducing fluid convection above the biological sample.

Provided herein are compositions useful for reducing convection flow around a biological sample. In some embodiments, a composition comprises a substrate comprising a capture area, a biological sample disposed on the capture area, and a buffer disposed on the biological sample; a gasket disposed on the substrate, wherein the gasket generates a chamber encompassing the lateral dimension of the capture area; and a porous insert inserted into the chamber and in contact with the buffer and/or the biological sample; wherein the porous insert defines a height of the gasketed area and limits free flow space in the gasketed area, wherein the substrate comprises a capture probe attached to the capture area, wherein the capture probe comprises a spatial barcode and a capture domain, and wherein the capture domain is hybridized directly or indirectly to a biological analyte from the biological sample or an intermediate agent indicative of the presence of the biological analyte. In some embodiments, a composition reduces fluid convection above the biological sample. In some embodiments, the buffer is in the porous insert.

In some embodiments of methods, compositions, and kits provided herein, a substrate comprises a capture probe attached to a capture area, wherein the capture probe comprises a spatial barcode and a capture domain, and wherein the capture domain hybridizes directly or indirectly to a biological analyte from the biological sample or an intermediate agent indicative of the presence of the biological analyte. In some embodiments, a capture probe comprises a unique molecular identifier (UMI).

In some embodiments, a biological sample is a fresh frozen tissue sample, and a capture domain hybridizes to an mRNA released from the cell; or, a biological sample is a formalin-fixed paraffin-embedded (FFPE) tissue sample, and the capture domain hybridizes to a ligation product that is a proxy for a target mRNA in the biological sample.

In some embodiments of methods, compositions, and kits provided herein, a porous insert comprises a hydrogel, a foam, a metal mesh, a porous ceramic, or a combination thereof. In some embodiments, the hydrogel comprises PEGDA poly(ethyleneglycol diacrylate), poly(hydroxyethyl methacrylate), agarose, alginate, poly(acrylamide), methylcellulose, collagen, gelatin, poly(acrylic acid), poly(ethyleneglycol dimethacrylate), poly(vinyl pyrrolidone), carboxymethyl cellulose, chitosan, poly(vinyl alcohol), chitin, carrageenan, or a combination thereof. In some embodiments, the poly(ethyleneglycol diacrylate) is about 30% to 50% of the hydrogel. In some embodiments, the foam comprises polyethylene, polyurethane, polystyrene, polyvinyl, rubber foam, thermoplastic elastomer foam, or a combination thereof. In some embodiments, the metal mesh comprises aluminum, brass, bronze, copper, steel, or a combination thereof. In some embodiments, the porous ceramic comprises silicate, diatomite, carbon, corundum, silicon carbide, ocordierite, or a combination thereof. A porous insert may contact a biological sample. A porous insert may be separated from a biological sample by a spacer. In some embodiments, a spacer has a thickness in a range from about 5 μm to about 20 μm. A porous insert may be immediately adjacent to a biological sample. In some embodiments, a buffer comprises a permeabilization enzyme. In some embodiments, a porous insert may be prefilled with a buffer for disposing on a biological sample. In some embodiments, a porous insert comprises a plurality of pores, each pore having a diameter in a range from about 10 nm to about 100 μm; or wherein the porous insert has a contact angle between about 0° and about 80°; or wherein the porous insert has a compressibility between about 1×10⁻⁵ m²/N and about 1×10⁻⁸ m²/N. In some embodiments, a porous insert is thermally stable at 37° C.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A is a schematic illustration of a composition or system useful for reducing fluid convection around a biological sample. The various components of the composition or system are shown, in addition to the biological sample that is disposed on a slide, such as a spatially arrayed area on a slide.

FIG. 1B is a schematic illustration showing that the insert can be inserted into the gasketed area of the spatial array, and into the buffer solution. The insert can make direct contact with the biological sample.

FIG. 1C is a schematic illustration showing that the insert can be removed from the gasketed area. The pores of the insert may extract any buffer solution present in the gasketed area upon removal.

FIG. 2A is a schematic illustration showing an additional design consideration for an insert of the disclosure. A porous insert may comprise a cap comprising a grip piece, useful for manipulation of the porous insert. The cap may facilitate pre-filling or post-filling of the porous insert with a buffer solution before or after inserting into the gasketed area.

FIG. 2B is a schematic illustration showing a design consideration of an insert comprising a cap, that can be snugly inserted into a gasketed area. The insert can make contact with the biological sample. The insert can subsequently be removed from the gasketed area.

FIG. 3A is a photograph of an insert with an attached spacer. A spacer can be attached to the bottom of an insert before insertion of the insert into a gasketed area such that the insert does not make direct contact with the biological sample.

FIG. 3B is a schematic illustration of an insert showing that an insert can have a shape that facilitates manipulation of the insert, such as by the use of tweezers or forceps.

FIG. 3C is a schematic illustration of a spacer. A spacer can have defined dimensions, such as those based on the dimensions of an associated insert.

FIG. 4A is a schematic illustration of an exemplary insert. An insert can have a defined width, depth, and height suitable for insertion into a chamber made by a gasket on a cassette in which is located an arrayed area, for example including a biological sample on the arrayed area.

FIG. 4B is a schematic illustration of an exemplary composition/system comprising an insert. An insert may be placed in a gasketed chamber and may be held in place by contact with an assay cassette upon which is located the gasketed chamber.

FIG. 5A shows the results of an experiment testing the effect of a porous insert on gene expression, using UMI counts as a measure of sensitivity. Shown is a quantification of medium UMI counts per spot under conditions of “No insert”, “Foam insert”, “50 m pore insert”, and “50 μm pore insert+spacer” following a 10 minute tissue permeabilization step.

FIG. 5B shows representative signal intensity heat-maps of mouse brain tissue samples from the FIG. 5A experiment showing the spatial location of UMI counts underneath and adjacent to a mouse brain tissue sample following a 10 minute tissue permeabilization step in conjunction with the different insert conditions.

### DETAILED DESCRIPTION

### I. Introduction

In current methodologies used to study the spatial distribution of a target analyte (e.g., target messenger RNA (mRNA)), a tissue is permeabilized by proteinase in a solution, and the tissue-associated mRNA transcripts are released and transported to an adjacent space by diffusion. Absent outside forces, mRNA transcripts are assumed to diffuse vertically out of the tissue by gravity, down to the surface of an array, such that they are captured proximal to where they were natively present in the tissue sample. However, outside forces that result in convection can create a discrepancy between transcripts' native position in tissue and their corresponding bound position on a detection or capture area (i.e., as on a spatial arrayed slide). This disclosure provides an engineered insert structure and a method to suppress convection above the tissue by limiting the free flow space, reducing the number of transcripts captured in the area surrounding a biological sample-associated region on a capture area adjacent to the biological sample (i.e., captured outside of a biological sample-associated region). An insert structure may be made of porous material, and may include a hydrophilic surface, which allow a permeabilization solution to easily penetrate through the insert structure while keeping the tissue wet during permeabilization.

Spatial profiling of one or more target analytes in a biological sample is subject to numerous physical and technical challenges. One challenge results from the isolation of a target analyte from native and/or processed tissue. After a biological sample is processed such that a target analyte is isolated from the biological sample, several current technologies require subsequent ex vivo capture of the target analyte. Some current technologies rely on the assumption that a target analyte does not undergo lateral displacement during the capture process (i.e., once the target analyte leaves its native position in tissue). However, various physical forces have the potential to cause mislocalization of a target analyte before capture. These forces include but are not limited to those that cause upward or lateral convection of fluid, causing a target analyte to move away from the lateral position at which the analyte exited the biological sample. Fluid convection may result from one or more of temperature gradients in an assay buffer, surface tension gradients, a high buffer volume, and leaks or air pockets in an assay chamber.

Provided herein are technologies useful for limiting the movement of a target analyte and subsequent mislocalization of said target analyte during spatial profiling. Provided solutions to these problems include but are not limited to technologies that reduce the reaction volume in an assay chamber or otherwise reduce the effects of system dynamics on the movement of a target analyte. These ends may be met through the use of an insert structure placed into an assay chamber to reduce the volume of the assay buffer. Other solutions include increasing the viscosity of a reaction buffer. The technologies provided herein are useful for enhancing the accuracy and resolution in spatial analyses of target analytes in a biological sample by reducing analyte mislocalization. The provided technologies greatly improve current approaches to biological spatial profiling and will enable researchers to gain much sought after insight into the spatial dynamics of biological systems, a rigorous understanding of which has been elusive.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte on a spatial array that correlates to a location within a cell or a tissue sample (e.g., mammalian cell or a mammalian tissue sample) and a capture domain that is capable of binding to or hybridizing to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent, or a nucleic acid that serves as a proxy for a target nucleic acid sequence. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

### II. General Definitions

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis.

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids.

Examples of nucleic acid analytes also include RNA analytes such as various types of coding and non-coding RNA Examples of the different types of RNA analytes include messenger RNA (mRNA), ribosomal RNA (rRNA), transfer RNA (tRNA), microRNA (miRNA), and viral RNA The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA The RNA can be circular RNA The RNA can be a bacterial rRNA (e.g., 16s rRNA or 23s rRNA).

Additional examples of analytes include mRNA and cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein). In some embodiments, a perturbation agent can be a small molecule, an antibody, a drug, an aptamer, a miRNA, a physical environmental (e.g., temperature change), or any other known perturbation agents.

Analytes can include a nucleic acid molecule with a nucleic acid sequence encoding at least a portion of a V(D)J sequence of an immune cell receptor (e.g., a TCR or BCR). In some embodiments, the nucleic acid molecule is cDNA first generated from reverse transcription of the corresponding mRNA, using a poly(T) containing primer. The generated cDNA can then be barcoded using a capture probe, featuring a barcode sequence (and optionally, a UMI sequence) that hybridizes with at least a portion of the generated cDNA In some embodiments, a template switching oligonucleotide hybridizes to a poly(C) tail added to a 3′ end of the cDNA by a reverse transcriptase enzyme. The original mRNA template and template switching oligonucleotide can then be denatured from the cDNA and the barcoded capture probe can then hybridize with the cDNA and a complement of the cDNA generated. Additional methods and compositions suitable for barcoding cDNA generated from mRNA transcripts including those encoding V(D)J regions of an immune cell receptor and/or barcoding methods and composition including a template switch oligonucleotide are described in PCT Patent Application PCT/US2017/057269, filed Oct. 18, 2017, and U.S. patent application Ser. No. 15/825,740, filed Nov. 29, 2017, both of which are incorporated herein by reference in their entireties. V(D)J analysis can also be completed with the use of one or more labelling agents that bind to particular surface features of immune cells and associated with barcode sequences. The one or more labelling agents can include an MHC or MHC multimer.

As described above, the analyte can include a nucleic acid capable of functioning as a component of a gene editing reaction, such as, for example, clustered regularly interspaced short palindromic repeats (CRISPR)-based gene editing. Accordingly, the capture probe can include a nucleic acid sequence that is complementary to the analyte (e.g., a sequence that can hybridize to the CRISPR RNA (crRNA), single guide RNA (sgRNA), or an adapter sequence engineered into a crRNA or sgRNA).

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample or can be obtained at intervals from a sample that continues to remain in viable condition.

In general, the systems, apparatus, methods, and compositions can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (1)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template. For example, a ligation product is formed by two probes that hybridize to sequences on a target nucleic acid. The two probes can be adjacently or non-adjacently hybridized to the target nucleic acid sequences. If hybridized adjacently, the two probes can be ligated together to form a ligation product which serves as a proxy for the target nucleic acid sequence. If hybridized non-adjacently, the 3′ end of one of the probes is extended to meet the 5′ end of the other probe, thus gap filling the distance between the two probes with nucleic acids complementary to the target nucleic acids in that region that is between, but not hybridized, to the two probes. Once the gap between the two probes is filled, the two probes can be ligated together to form a ligation product that serves as a proxy for the target nucleic acid. The ligation product can further include functional sequences for use in spatial array methods. For example, one of the probes includes a sequence that is compatible to a next generation sequencing workflow (e.g., a Read 1 or Read 2 sequence if using and Illumina sequencing instrument). Additionally, one of the probes includes a sequence that is complementary to the capture domain of a capture probe on the spatial array, such that the ligation product can be captured by a capture probe on the spatial array.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

The terms “hybridizing,” “hybridize,” “annealing,” and “anneal” are used interchangeably in this disclosure and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

In some embodiments, the quantification of RNA and/or DNA is carried out by real-time PCR (also known as quantitative PCR or qPCR), using techniques well known in the art, such as but not limited to “TAQMAN™”, or dyes such as “SYBR®”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

### III. Spatial Analysis Methods

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (in particular an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### IV. Transcript Mislocalization (TML) Mitigation

Provided herein are technologies to reduce the mislocalization of a target analyte (e.g., mRNA transcript) during a spatial analysis assay as described above.

In some embodiments, a method for reducing fluid convection comprises providing (1) a substrate comprising a capture area, a biological sample comprising a cell or a plurality of cells disposed on the capture area, a buffer disposed on the biological sample, and a gasket disposed on the substrate, wherein the gasket provides a chamber comprising the lateral dimension of the capture area (including the fiducial marks that surround the capture area) and includes a height above the capture area and biological sample; and (2) a porous insert positioned in the chamber and in contact with the buffer and/or the biological sample, wherein the porous insert limits free flow space in the chamber, thereby reducing fluid convection above the biological sample.

In some embodiments, a method for reducing fluid convection comprises (1) providing a substrate comprising a capture area, a biological sample comprising a cell disposed on the capture area, and a gasket disposed on the substrate, wherein the gasket comprises an opening encompassing the lateral dimension of the capture area (including the fiducial marks that surround the capture area) and includes a height above the capture area and biological sample; and (2) inserting a porous insert in the opening, wherein the porous insert is pre-loaded with a buffer, wherein the porous insert limits free flow space of the buffer pre-loaded in the porous insert, thereby reducing fluid convection above the biological sample.

### Biological Samples

A biological sample can be obtained from a subject for analysis using any of a variety of techniques including, but not limited to, biopsy, surgery, and laser capture microscopy (LCM), and generally includes cells and/or other biological material from the subject. In addition to the subjects described above, a biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include fetal cells. For example, a procedure such as amniocentesis can be performed to obtain a fetal cell sample from maternal circulation. Sequencing of fetal cells can be used to identify any of a number of genetic disorders, including, e.g., aneuploidy such as Down's syndrome, Edwards syndrome, and Patau syndrome. Further, cell surface features of fetal cells can be used to identify any of a number of disorders or diseases.

Biological samples can also include immune cells. Sequence analysis of the immune repertoire of such cells, including genomic, proteomic, and cell surface features, can provide a wealth of information to facilitate an understanding the status and function of the immune system By way of example, determining the status (e.g., negative or positive) of minimal residue disease (MRD) in a multiple myeloma (MM) patient following autologous stem cell transplantation is considered a predictor of MRD in the MM patient (see, e.g., U.S. Patent Application Publication No. 2018/0156784, the entire contents of which are incorporated herein by reference).

Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

As discussed above, a biological sample can include a single analyte of interest, or more than one analyte of interest.

### Biological Sample Preparation

A variety of steps can be performed to prepare a biological sample for analysis. Except where indicated otherwise, the preparative steps described below can generally be combined in any manner to appropriately prepare a particular sample for analysis.

### (1) Tissue Sectioning

A biological sample can be harvested from a subject (e.g., via surgical biopsy, whole subject sectioning), grown in vitro on a growth substrate or culture dish as a population of cells, or prepared as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., about 5-20 micrometers thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 micrometers. Thicker sections can also be used if desired or convenient, e.g., at least 70, 80, 90, or 100 micrometers or more. Typically, the thickness of a tissue section is between 1-100 micrometers, 1-50 micrometers, 1-30 micrometers, 1-25 micrometers, 1-20 micrometers, 1-15 micrometers, 1-10 micrometers, 2-8 micrometers, 3-7 micrometers, or 4-6 micrometers, but as mentioned above, sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to build a more three-dimensional like picture of gene expression for the tissue as a whole.

### ) (2) Freezing

In some embodiments, the biological sample (e.g., a tissue section as described above) can be prepared by deep freezing at a temperature suitable to maintain or preserve the integrity (e.g., the physical characteristics) of the tissue structure. Such a temperature can be, e.g., less than −20° C., or less than −25° C., −30° C., −40° C., −50° C., −60° C., −70° C., 80° C. −90° C., −100° C., −110° C., −120° C., −130° C., −140° C., −150° C., −160° C., −170° C., −180° C., −190° C., or −200° C. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C. A sample can be snap frozen in isopentane and liquid nitrogen. Frozen samples can be stored in a sealed container prior to embedding.

### (3) Formalin Fixation and Paraffin Embedding

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

### (4) Fixation

As an alternative to formalin fixation described above, a biological sample can be fixed in any of a variety of other fixatives to preserve the biological structure of the sample prior to analysis. For example, a sample can be fixed via immersion in ethanol, methanol, acetone, formaldehyde (e.g., 2% formaldehyde), paraformaldehyde-Triton, glutaraldehyde, or combinations thereof.

In some embodiments, acetone fixation is used with fresh frozen samples, which can include, but are not limited to, cortex tissue, mouse olfactory bulb, human brain tumor, human post-mortem brain, and breast cancer samples. In some embodiments, a compatible fixation method is chosen and/or optimized based on a desired workflow. For example, formaldehyde fixation may be chosen as compatible for workflows using IHC/IF protocols for protein visualization. As another example, methanol fixation may be chosen for workflows emphasizing RNA/DNA library quality. Acetone fixation may be chosen in some applications to permeabilize the tissue. When acetone fixation is performed, pre-permeabilization steps (described below) may not be performed. Alternatively, acetone fixation can be performed in conjunction with permeabilization steps.

### (5) Embedding

As an alternative to paraffin embedding described above, a biological sample can be embedded in any of a variety of other embedding materials to provide a substrate to the sample prior to sectioning and other handling steps. In general, the embedding material is removed prior to analysis of tissue sections obtained from the sample. Suitable embedding materials include, but are not limited to, Optimal Cutting Temperature compound, waxes, resins (e.g., methacrylate resins), epoxies, and agar.

### (6) Staining

To facilitate visualization, biological samples can be stained using a wide variety of stains and staining techniques. In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin.

The sample can be stained using known staining techniques, including Can-Grunwald, Giemsa, hematoxylin and eosin (H&E), JenneR1s, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other low pH acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in a low pH acid (e.g., HCl). In some embodiments, destaining can include adding HCl to a downstream solution (e.g., permeabilization solution). In some embodiments, destaining can include dissolving an enzyme used in the disclosed methods (e.g., pepsin) in a low pH acid (e.g., HCl) solution. In some embodiments, after destaining hematoxylin with a low pH acid, other reagents can be added to the destaining solution to raise the pH for use in other applications. For example, SDS can be added to a low pH acid destaining solution in order to raise the pH as compared to the low pH acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J Histochem. Cytochem. 2017; 65(8): 431-444, Lin et al., Nat Commun. 2015; 6:8390, Pirici et al., J Histochem. Cytochem. 2009; 57:567-75, and Glass et al., J Histochem. Cytochem. 2009; 57:899-905, the entire contents of each of which are incorporated herein by reference.

### (7) Hydrogel Embedding

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus. A “hydrogel” as described herein can include a cross-linked 3D network of hydrophilic polymer chains. A “hydrogel subunit” can be a hydrophilic monomer, a molecular precursor, or a polymer that can be polymerized (e.g., cross-linked) to form a three-dimensional (3D) hydrogel network.

A hydrogel can swell in the presence of water. In some embodiments, a hydrogel comprises a natural material. In some embodiments, a hydrogel includes a synthetic material. In some embodiments, a hydrogel includes a hybrid material, e.g., the hydrogel material comprises elements of both synthetic and natural polymers. Any of the materials used in hydrogels or hydrogels comprising a polypeptide-based material described herein can be used. Embedding the sample in this manner typically involves contacting the biological sample with a hydrogel such that the biological sample becomes surrounded by the hydrogel. For example, the sample can be embedded by contacting the sample with a suitable polymer material and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other hydrogel-formation method known in the art. For example, the biological sample can be immobilized in the hydrogel by polyacrylamide crosslinking. Further, analytes of a biological sample can be immobilized in a hydrogel by crosslinking (e.g., polyacrylamide crosslinking).

The composition and application of the hydrogel to a biological sample typically depends on the nature and preparation of the biological sample (e.g., sectioned, non-sectioned, fresh-frozen tissue, type of fixation). A hydrogel can be any appropriate hydrogel where upon formation of the hydrogel the biological sample becomes anchored to or embedded in the hydrogel. Non-limiting examples of hydrogels are described herein or are known in the art. As one example, where the biological sample is a tissue section, the hydrogel can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, a hydrogel is formed in compartments, including but not limited to devices used to culture, maintain, or transport the cells. For example, hydrogels can be formed with monomer solution plus APS/TEMED added to the compartment to a depth ranging from about 0.1 μm to about 5 mm.

In some embodiments, the hydrogel can be embedded in the pores of an insert that is applied to a biological sample. For example, a hydrogel could be embedded into the pores of a plastic insert, a ceramic insert or a metal insert.

In some embodiments, a hydro gel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE) (available from ThermoFisher, Waltham, MA), Label-IT Amine (available from MirusBio, Madison, WI) and Label X (Chen et al., Nat. Methods 13:679-684, (2016)).

In some embodiments, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., capture probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell tagging agent is added to the hydrogel before, contemporaneously with, and/or after polymerization. In some embodiments, a cell-penetrating agent is added to the hydrogel before, contemporaneously with, and/or after polymerization.

In some embodiments, a biological sample is embedded in a hydrogel to facilitate sample transfer to another location (e.g., to an array). For example, archived biological samples (e.g., FFPE tissue sections) can be transferred from storage to a spatial array to perform spatial analysis. In some embodiments, a biological sample on a substrate can be covered with any of the prepolymer solutions described herein. In some embodiments, the prepolymer solution can be polymerized such that a hydrogel is formed on top of and/or around the biological sample. Hydrogel formation can occur in a manner sufficient to anchor (e.g., embed) the biological sample to the hydrogel. After hydrogel formation, the biological sample is anchored to (e.g., embedded in) the hydrogel wherein separating the hydrogel from the substrate (e.g., glass slide) results in the biological sample separating from the substrate along with the hydrogel. The biological sample contained in the hydrogel can then be contacted with a spatial array, and spatial analysis can be performed on the biological sample.

Any variety of characteristics can determine the transfer conditions required for a given biological sample. Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

In some embodiments, the hydrogel is removed after contacting the biological sample with the spatial array. For example, methods described herein can include an event-dependent (e.g., light or chemical) depolymerizing hydrogel, wherein upon application of the event (e.g., external stimuli) the hydrogel depolymerizes. In one example, a biological sample can be anchored to a DTT-sensitive hydrogel, where addition of DTT can cause the hydrogel to depolymerize and release the anchored biological sample.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored in a medium before or after clearing of hydrogel (e.g., a mounting medium, methylcellulose, or other semi-solid mediums).

In some embodiments, the hydrogel chemistry can be tuned to specifically bind (e.g., retain) particular species of analytes (e.g., RNA, DNA, protein, etc.). In some embodiments, a hydrogel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE), Label-IT Amine and Label X (Chen et al., Nat. Methods 13:679-684, (2016)). Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)).

Additional methods and aspects of hydrogel embedding of biological samples are described for example in Chen et al., Science 347(6221):543-548, 2015, the entire contents of which are incorporated herein by reference.

### (8) Biological Sample Transfer

In some embodiments, a biological sample immobilized on a substrate (e.g., a biological sample prepared using methanol fixation or formalin-fixation and paraffin-embedding (FFPE)) is transferred to a spatial array using a hydrogel. In some embodiments, a hydrogel is formed on top of a biological sample on a substrate (e.g., glass slide). For example, hydrogel formation can occur in a manner sufficient to anchor (e.g., embed) the biological sample to the hydrogel. After hydrogel formation, the biological sample is anchored to (e.g., embedded in) the hydrogel wherein separating the hydrogel from the substrate results in the biological sample separating from the substrate along with the hydrogel. The biological sample can then be contacted with a spatial array, thereby allowing spatial profiling of the biological sample. In some embodiments, the hydrogel is removed after contacting the biological sample with the spatial array. For example, methods described herein can include an event-dependent (e.g., light or chemical) depolymerizing hydrogel, wherein upon application of the event (e.g., external stimuli) the hydrogel depolymerizes. In one example, a biological sample can be anchored to a DTT-sensitive hydrogel, where addition of DTT can cause the hydrogel to depolymerize and release the anchored biological sample. A hydrogel can be any appropriate hydrogel where upon formation of the hydrogel on the biological sample the biological sample becomes anchored to or embedded in the hydrogel. Non-limiting examples of hydrogels are described herein or are known in the art. In some embodiments, a hydrogel includes a linker that allows anchoring of the biological sample to the hydrogel. In some embodiments, a hydrogel includes linkers that allow anchoring of biological analytes to the hydrogel. In such cases, the linker can be added to the hydrogel before, contemporaneously with, or after hydrogel formation. Non-limiting examples of linkers that anchor nucleic acids to the hydrogel can include 6-((Acryloyl)amino) hexanoic acid (Acryloyl-X SE) (available from ThermoFisher, Waltham, MA), Label-IT Amine (available from MirusBio, Madison, WI) and Label X (Chen et al., Nat. Methods 13:679-684, 2016). Any variety of characteristics can determine the transfer conditions required for a given biological sample. Non-limiting examples of characteristics likely to impact transfer conditions include the sample (e.g., thickness, fixation, and cross-linking) and/or the analyte of interest (different conditions to preserve and/or transfer different analytes (e.g., DNA, RNA, and protein)). In some embodiments, hydrogel formation can occur in a manner sufficient to anchor the analytes (e.g., embed) in the biological sample to the hydrogel. In some embodiments, the hydrogel can be imploded (e.g., shrunk) with the anchored analytes (e.g., embedded in the hydrogel) present in the biological sample. In some embodiments, the hydrogel can be expanded (e.g., isometric expansion) with the anchored analytes (e.g., embedded in the hydrogel) present in the biological sample. In some embodiments, the hydrogel can be imploded (e.g., shrunk) and subsequently expanded with anchored analytes (e.g., embedded in the hydrogel) present in the biological sample.

### (9) Isometric Expansion

In some embodiments, a biological sample embedded in a hydrogel can be isometrically expanded. Isometric expansion methods that can be used include hydration, a preparative step in expansion microscopy, as described in Chen et al., Science 347(6221):543-548, 2015; Asano et al. Current Protocols. 2018, 80:1, doi:10.1002/cpcb.56 and Gao et al. BMC Biology. 2017, 15:50, doi:10.1186/s12915-017-0393-3, Wassie, A T., et al, Expansion microscopy: principles and uses in biological research, Nature Methods, 16(1): 33-41 (2018), each of which is incorporated by reference in its entirety.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

Isometric expansion can be performed by anchoring one or more components of a biological sample to a gel, followed by gel formation, proteolysis, and swelling. Isometric expansion of the biological sample can occur prior to immobilization of the biological sample on a substrate, or after the biological sample is immobilized to a substrate. In some embodiments, the isometrically expanded biological sample can be removed from the substrate prior to contacting the expanded biological sample with a spatially barcoded array (e.g., spatially barcoded capture probes on a substrate).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. For example, isometric expansion of the biological sample can result in increased resolution in spatial profiling (e.g., single-cell profiling). The increased resolution in spatial profiling can be determined by comparison of an isometrically expanded sample with a sample that has not been isometrically expanded.

Isometric expansion can enable three-dimensional spatial resolution of the subsequent analysis of the sample. In some embodiments, isometric expansion of the biological sample can occur in the presence of spatial profiling reagents (e.g., analyte capture agents or capture probes). For example, the swellable gel can include analyte capture agents or capture probes anchored to the swellable gel via a suitable linker. In some embodiments, spatial profiling reagents can be delivered to particular locations in an isometrically expanded biological sample.

In some embodiments, a biological sample is isometrically expanded to a volume at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded volume. In some embodiments, the sample is isometrically expanded to at least 2× and less than 20× of its non-expanded volume.

In some embodiments, a biological sample embedded in a hydrogel is isometrically expanded to a volume at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded volume. In some embodiments, the biological sample embedded in a hydrogel is isometrically expanded to at least 2× and less than 20× of its non-expanded volume.

### (10) Substrate Attachment

In some embodiments, the biological sample can be attached to a substrate. Examples of substrates suitable for this purpose are described in detail below. Attachment of the biological sample can be irreversible or reversible, depending upon the nature of the sample and subsequent steps in the analytical method.

In certain embodiments, the sample can be attached to the substrate reversibly by applying a suitable polymer coating to the substrate and contacting the sample to the polymer coating. The sample can then be detached from the substrate using an organic solvent that at least partially dissolves the polymer coating. Hydrogels are examples of polymers that are suitable for this purpose.

More generally, in some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

### (11) Unaggregated Cells

In some embodiments, the biological sample corresponds to cells (e.g., derived from a cell culture or a tissue sample). In a cell sample with a plurality of cells, individual cells can be naturally unaggregated. For example, cells can be derived from a suspension of cells and/or disassociated or disaggregated cells from a tissue or tissue section.

Alternatively, the cells in the sample may be aggregated, and may be disaggregated into individual cells using, for example, enzymatic or mechanical techniques. Examples of enzymes used in enzymatic disaggregation include, but are not limited to, dispase, collagenase, trypsin, or combinations thereof. Mechanical disaggregation can be performed, for example, using a tissue homogenizer.

In some embodiments of unaggregated cells or disaggregated cells, the cells are distributed onto the substrate such that at least one cell occupies a distinct spatial feature on the substrate. The cells can be immobilized on the substrate (e.g., to prevent lateral diffusion of the cells). In some embodiments, a cell immobilization agent can be used to immobilize a non-aggregated or disaggregated sample on a spatially-barcoded array prior to analyte capture. A “cell immobilization agent” can refer to an antibody, attached to a substrate, which can bind to a cell surface marker. In some embodiments, the distribution of the plurality of cells on the substrate follows Poisson statistics.

In some embodiments, cells from a plurality of cells are immobilized on a substrate. In some embodiments, the cells are immobilized to prevent lateral diffusion, for example, by adding a hydrogel and/or by the application of an electric field.

### (12) Suspended and Adherent Cells

In some embodiments, the biological sample can be derived from a cell culture grown in vitro. Samples derived from a cell culture can include one or more suspension cells which are anchorage-independent within the cell culture. Examples of such cells include, but are not limited to, cell lines derived from hematopoietic cells, and from the following cell lines: Colo205, CCRF-CEM, HL-60, K562, MOLT-4, RPMI-8226, SR, HOP-92, NCI-H322M, and MALME-3M.

Samples derived from a cell culture can include one or more adherent cells which grow on the surface of the vessel that contains the culture medium Non-limiting examples of adherent cells include DU145 (prostate cancer) cells, H295R (adrenocortical cancer) cells, HeLa (cervical cancer) cells, KBM-7 (chronic myelogenous leukemia) cells, LNCaP (prostate cancer) cells, MCF-7 (breast cancer) cells, MDA-MB-468 (breast cancer) cells, PC3 (prostate cancer) cells, SaOS-2 (bone cancer) cells, SH-SY5Y (neuroblastoma, cloned from a myeloma) cells, T-47D (breast cancer) cells, THP-1 (acute myeloid leukemia) cells, U87 (glioblastoma) cells, National Cancer Institute's 60 cancer cell line panel (NCT60), vero (African green monkey Chlorocebus kidney epithelial cell line) cells, MC3T3 (embryonic calvarium) cells, GH3 (pituitary tumor) cells. PC12 (pheochromocytoma) cells, dog MDCK kidney epithelial cells, Xenopus A6 kidney epithelial cells, zebrafish AB9 cells, and Sf9 insect epithelial cells.

Additional examples of adherent cells are shown in Table 1 and catalogued, for example, in “A Catalog of in Vitro Cell Lines, Transplantable Animal and Human Tumors and Yeast,” The Division of Cancer Treatment and Diagnosis (DCTD), National Cancer Institute (2013), and in Abaan et al., “The exomes of the NCI-60 panel: a genomic resource for cancer biology and systems pharmacology,” Cancer Research 73(14):4372-82, 2013, the entire contents of each of which are incorporated by reference herein.

In some embodiments, the adherent cells are cells that correspond to one or more of the following cell lines: BT549, HS 578T, MCF7, MDA-MB-231, MDA-MB-468, T-47D, SF268, SF295, SF539, SNB-19, SNB-75, U251, Colo205, HCC 2998, HCT-116, HCT-15, HT29, KM12, SW620, 786-0, A498, ACHN, CAKI, RXF 393, SN12C, TK-10, U0-31, A549, EKVX, HOP-62, HOP-92, NCI-H226, NCI-H23, NCI-H460, NCI-H522, LOX IMVI, M14, MALME-3M, MDA-MB-435, SK-, EL-2, SK-MEL-28, SK-MEL-5, UACC-257, UACC-62, IGROV1, OVCAR-3, OVCAR-4, OVCAR-5, OVCAR-8, SK-OV-3, NCI-ADR-RES, DU145, PC-3, DU145, H295R, HeLa, KBM-7, LNCaP, MCF-7, MDA-MB-468, PC3, SaOS-2, SH-5Y5Y, T-47D, THP-1, U87, vero, MC3T3, GH3, PC12, dog MDCK kidney epithelial, Xenopus A6 kidney epithelial, zebrafish AB9, and Sf9 insect epithelial cell lines.

### (13) Tissue Permeabilization

In some embodiments, a biological sample can be permeabilized to facilitate transfer of analytes out of the sample, and/or to facilitate transfer of species (such as capture probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20Tm, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). In some embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)).

In some embodiments, a biological sample can be permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 WIT %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, a biological sample can be permeabilized by exposing the sample (e.g., for about 5 minutes to about 1 hour, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 WA, % to about 14.0 w/v %, about 2.0 w/v % to about 12.0 w/v %, about 2.0 w/v % to about 10.0 w/v %, about 4.0 w/v ( )/0 to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v°/0 to about 14.0 w/v %) SDS and/or N-lauroylsarcosine salt solution and/or proteinase K (e.g., at a temperature of about 4% to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In some embodiments, the biological sample can be incubated with a permeabilizing agent to facilitate permeabilization of the sample. Additional methods for sample permeabilization are described, for example, in Jamur et al., Method Mol. Biol. 588:63-66, 2010, the entire contents of which are incorporated herein by reference.

In some embodiments, the biological sample can be permeabilized by adding one or more surfactants to the permeabilization solution. For example, surfactant-based solutions can be used to permeabilize cells. Permeabilization solutions can include ionic surfactants such as, for example, sarkosyl and sodium dodecyl sulfate (SDS). More generally, additional permeabilization agents can include, without limitation, organic solvents, chelating agents, detergents, surfactants, and chaotropic agents.

### Proteases

In some embodiments, a medium, solution, or permeabilization solution may contain one or more enzymes (i.e., one or more permeabilization enzymes). In some embodiments, a medium, solution, or permeabilization solution may contain one or more proteases. In some embodiments, a biological sample treated with a protease capable of degrading histone proteins can result in the generation of fragmented genomic DNA. The fragmented genomic DNA can be captured using the same capture domain (e.g., capture domain having a poly(T) sequence) used to capture mRNA. In some embodiments, a biological sample is treated with a protease capable of degrading histone proteins and an RNA protectant prior to spatial profiling in order to facilitate the capture of both genomic DNA and mRNA.

In some embodiments, a biological sample is permeabilized by exposing the sample to a protease capable of degrading histone proteins. As used herein, the term “histone protein” typically refers to a linker histone protein (e.g., H1) and/or a core histone protein (e.g., H2A, H2B, H3, and H4). In some embodiments, a protease degrades linker histone proteins, core histone proteins, or linker histone proteins and core histone proteins. Any suitable protease capable of degrading histone proteins in a biological sample can be used. Non-limiting examples of proteases capable of degrading histone proteins include proteases inhibited by leupeptin and TLCK (Tosyl-L-lysyl-chloromethane hydrochloride), a protease encoded by the EUO gene from Chlamydia trachomatis serovar A, granzyme A, a serine protease (e.g., trypsin or trypsin-like protease, neutral serine protease, elastase, cathepsin G), an aspartyl protease (e.g., cathepsin D), a peptidase family Cl enzyme (e.g., cathepsin L), pepsin, proteinase K, a protease that is inhibited by the diazomethane inhibitor Z-Phe-Phe-CHN(2) or the epoxide inhibitor E-64, a lysosomal protease, or an azurophilic enzyme (e.g., cathepsin G, elastase, proteinase 3, neutral serine protease). In some embodiments, a serine protease is a trypsin enzyme, trypsin-like enzyme or a functional variant or derivative thereof (e.g., P00761; COHK48; Q8IYP2; Q8BW11; Q61E06; P35035; P00760; P06871; Q90627; P16049; P07477; P00762; P35031; P19799; P35036; Q29463; P06872; Q90628; P07478; P07146; P00763; P35032; P70059; P29786; P35037; Q90629; P35030; P08426; P35033; P35038; P12788; P29787; P35039; P35040; Q8NHM4; P35041; P35043; P35044; P54624; P04814; P35045; P32821; P54625; P35004; P35046; P32822; P35047; COHKAS; COHKA2; P54627; P35005; COHKA6; COHKA3; P52905; P83348; P00765; P35042; P81071; P35049; P51588; P35050; P35034; P35051; P24664; P35048; P00764; P00775; P54628; P42278; P54629; P42279; Q91041; P54630; P42280; COHKA4) or a combination thereof. In some embodiments, a trypsin enzyme is P00761, P00760, Q29463, or a combination thereof. In some embodiments, a protease capable of degrading one or more histone proteins comprises an amino acid sequence with at least 80% sequence identity to P00761, P00760, or Q29463. In some embodiments, a protease capable of degrading one or more histone proteins comprises an amino acid sequence with at least 85%, 90%, 91%, 92%, 93%, 94%, 95%, 96%, 97%, 98%, or 99% identity to P00761, P00760, or Q29463. A protease may be considered a functional variant if it has at least 50% e.g., at least 60%, 70%, 80%, 90%, 95%, 96%, 97%, 98%, 99%, or 100% of the activity relative to the activity of the protease in condition optimum for the enzyme. In some embodiments, the enzymatic treatment with pepsin enzyme, or pepsin like enzyme, can include: P03954/PEPA1_MACFU; P28712/PEPA1 RABIT; P27677/PEPA2 MACFU; P27821/PEPA2 RABIT; PODJD8/PEPA3HUMAN; P27822/PEPA3RABIT; PODJD7/PEPA4 HUMAN; P27678/PEPA4 MACFU; P28713/PEPA4 RABIT; PODJD9/PEPAS HUMAN; Q9D106/PEPA5MOUSE; P27823/PEP AF_RABIT; P00792/PEPABOVIN; Q9N2D4/PEPA_CALJA; Q9GMY6/PEPA_CANLF; P00793/PEPA_CHICK; P11489/PEPA MACMU; P00791/PEPA PIG; Q9GMY7/PEPARHIFE; Q9GMY8/PEPA SORUN; P81497/PEPA SUNMU; P13636/PEPA URSTH and functional variants and derivatives thereof, or a combination thereof. In some embodiments, the pepsin enzyme can include: P00791/PEPA PIG; P00792/PEPABOVIN, functional variants, derivatives, or combinations thereof.

Additionally, the protease may be contained in a reaction mixture (solution), which also includes other components (e.g., buffer, salt, chelator (e.g., EDTA), and/or detergent (e.g., SDS, N-Lauroylsarcosine sodium salt solution)). The reaction mixture may be buffered, having a pH of about 6.5-8.5, e.g., about 7.0-8.0. Additionally, the reaction mixture may be used at any suitable temperature, such as about 10-50° C., e.g., about 10-44° C., 11-43° C., 12-42° C., 13-41° C., 14-40° C., 15-39° C., 16-38° C., 17-37° C., e.g., about 10° C., 12° C., 15° C., 18° C., 20° C., 22° C., 25° C., 28° C., 30° C., 33° C., 35° C. or 37° C., preferably about 35-45° C., e.g., about 37° C.

### Other Reagents

In some embodiments, a permeabilization solution can contain additional reagents or a biological sample may be treated with additional reagents in order to optimize biological sample permeabilization. In some embodiments, an additional reagent is an RNA protectant. As used herein, the term “RNA protectant” typically refers to a reagent that protects RNA from RNA nucleases (e.g., RNases). Any appropriate RNA protectant that protects RNA from degradation can be used. A non-limiting example of a RNA protectant includes organic solvents (e.g., at least 60%, 65%, 70%, 75%, 80%, 85%, 90%, or 95% v/v organic solvent), which include, without limitation, ethanol, methanol, propan-2-ol, acetone, trichloroacetic acid, propanol, polyethylene glycol, acetic acid, or a combination thereof. In some embodiments, a RNA protectant includes ethanol, methanol and/or propan-2-ol, or a combination thereof. In some embodiments, a RNA protectant includes RNAlater ICE (ThermoFisher Scientific). In some embodiments, the RNA protectant comprises at least about 60% ethanol. In some embodiments, the RNA protectant comprises about 60-95% ethanol, about 0-35% methanol and about 0-35% propan-2-ol, wherein the total amount of organic solvent in the medium is not more than about 95%. In some embodiments, the RNA protectant comprises about 60-95% ethanol, about 5-20% methanol and about 5-20% propan-2-ol, wherein the total amount of organic solvent in the medium is not more than about 95%. In some embodiments, the RNA protectant includes a salt. The salt may include ammonium sulfate, ammonium bisulfate, ammonium chloride, ammonium acetate, cesium sulfate, cadmium sulfate, cesium iron (11) sulfate, chromium (111) sulfate, cobalt (11) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate and zinc sulfate. In some embodiments, the salt is a sulfate salt, for example, ammonium sulfate, ammonium bisulfate, cesium sulfate, cadmium sulfate, cesium iron (II) sulfate, chromium (III) sulfate, cobalt (II) sulfate, copper (II) sulfate, lithium sulfate, magnesium sulfate, manganese sulfate, potassium sulfate, sodium sulfate, or zinc sulfate. In some embodiments, the salt is ammonium sulfate. The salt may be present at a concentration of about 20 g/100 ml of medium or less, such as about 15 g/100 ml, 10 g/100 ml, 9 g/100 ml, 8 g/100 ml, 7 g/100 ml, 6 g/100 ml, 5 g/100 ml or less, e.g., about 4 g, 3 g, 2 g or 1 g/100 ml.

Additionally, the RNA protectant may be contained in a medium that further includes a chelator (e.g., EDTA), a buffer (e.g., sodium citrate, sodium acetate, potassium citrate, or potassium acetate, preferably sodium acetate), and/or buffered to a pH between about 4-8 (e.g., about 5).

In some embodiments, the biological sample is treated with one or more RNA protectants before, contemporaneously with, or after permeabilization. For example, a biological sample is treated with one or more RNA protectants prior to treatment with one or more permeabilization reagents (e.g., one or more proteases). In another example, a biological sample is treated with a solution including one or more RNA protectants and one or more permeabilization reagents (e.g., one or more proteases). In yet another example, a biological sample is treated with one or more RNA protectants after the biological sample has been treated with one or more permeabilization reagents (e.g., one or more proteases). In some embodiments, a biological sample is treated with one or more RNA protectants prior to fixation.

In some embodiments, identifying the location of the captured analyte in the biological sample includes a nucleic acid extension reaction. In some embodiments where a capture probe captures a fragmented genomic DNA molecule, a nucleic acid extension reaction includes DNA polymerase. For example, a nucleic acid extension reaction includes using a DNA polymerase to extend the capture probe that is hybridized to the captured analyte (e.g., fragmented genomic DNA) using the captured analyte (e.g., fragmented genomic DNA) as a template. The product of the extension reaction includes a spatially-barcoded analyte (e.g., spatially-barcoded fragmented genomic DNA). The spatially-barcoded analyte (e.g., spatially-barcoded fragmented genomic DNA) can be used to identify the spatial location of the analyte in the biological sample. Any DNA polymerase that is capable of extending the capture probe using the captured analyte as a template can be used for the methods described herein. Non-limiting examples of DNA polymerases include T7 DNA polymerase; Bsu DNA polymerase; and E. coli DNA Polymerase pol I.

### Diffusion-Resistant Media

In some embodiments, a diffusion resistant medium can be 10-5000 μm thick. In some embodiments, a diffusion-resistant medium, typically used to limit diffusion of analytes, can include at least one permeabilization reagent. For example, the diffusion-resistant medium (e.g., a hydrogel) can include wells (e.g., micro-, nano-, or picowells or pores) containing a permeabilization buffer or reagents. In some embodiments, the diffusion-resistant medium (e.g., a hydrogel) is soaked in permeabilization buffer prior to contacting the hydrogel with a sample. In some embodiments, the hydrogel or other diffusion-resistant medium can contain dried reagents or monomers to deliver permeabilization reagents when the diffusion-resistant medium is applied to a biological sample. In some embodiments, the diffusion-resistant medium, (e.g., hydrogel) is covalently attached to a solid substrate (e.g., an acrylated glass slide).

In some embodiments, the hydrogel can be modified to both deliver permeabilization reagents and contain capture probes. For example, a hydrogel film can be modified to include spatially-barcoded capture probes. The spatially-barcoded hydrogel film is then soaked in permeabilization buffer before contacting the spatially-barcoded hydrogel film to the sample. In another example, a hydrogel can be modified to include spatially-barcoded capture probes and designed to serve as a porous membrane (e.g., a permeable hydrogel) when exposed to permeabilization buffer or any other biological sample preparation reagent. The permeabilization reagent diffuses through the spatially-barcoded permeable hydrogel and permeabilizes the biological sample on the other side of the hydrogel. The analytes then diffuse into the spatially-barcoded hydrogel after exposure to permeabilization reagents. In such cases, the spatially-barcoded hydrogel (e.g., porous membrane) is facilitating the diffusion of the biological analytes in the biological sample into the hydrogel. In some embodiments, biological analytes diffuse into the hydrogel before exposure to permeabilization reagents (e.g., when secreted analytes are present outside of the biological sample or in instances where a biological sample is lysed or permeabilized by other means prior to addition of permeabilization reagents). In some embodiments, the permeabilization reagent is flowed over the hydrogel at a variable flow rate (e.g., any flow rate that facilitates diffusion of the permeabilization reagent across the spatially-barcoded hydrogel). In some embodiments, the permeabilization reagents are flowed through a microfluidic chamber or channel over the spatially-barcoded hydrogel. In some embodiments, after using flow to introduce permeabilization reagents to the biological sample, biological sample preparation reagents can be flowed over the hydrogel to further facilitate diffusion of the biological analytes into the spatially-barcoded hydrogel. The spatially-barcoded hydrogel film thus delivers permeabilization reagents to a sample surface in contact with the spatially-barcoded hydrogel, enhancing analyte migration and capture. In some embodiments, the spatially-barcoded hydrogel is applied to a sample and placed in a permeabilization bulk solution. In some embodiments, the hydrogel film soaked in permeabilization reagents is sandwiched between a sample and a spatially-barcoded array. In some embodiments, target analytes are able to diffuse through the permeabilizing reagent-soaked hydrogel and hybridize or bind the capture probes on the other side of the hydrogel. In some embodiments, the thickness of the hydrogel is proportional to the resolution loss. In some embodiments, wells (e.g., micro-, nano-, or picowells) can contain spatially-barcoded capture probes and permeabilization reagents and/or buffer. In some embodiments, spatially-barcoded capture probes and permeabilization reagents are held between spacers. In some embodiments, the sample is punch, cut, or transferred into the well, wherein a target analyte diffuses through the permeabilization reagent/buffer and to the spatially-barcoded capture probes. In some embodiments, resolution loss may be proportional to gap thickness (e.g., the amount of permeabilization buffer between the sample and the capture probes). In some embodiments, the diffusion-resistant medium (e.g.; hydrogel) is between approximately 50-500 micrometers thick including 500, 450, 400, 350, 300, 250, 200, 150, 100, or 50 micrometers thick, or any thickness within 50 and 500 micrometers.

In some embodiments, a biological sample is exposed to a porous membrane (e.g., a permeable membrane) to aid in permeabilization and limit diffusive analyte losses, while allowing permeabilization reagents to reach a sample. Membrane chemistry and pore volume can be manipulated to minimize analyte loss. In some embodiments, the porous membrane may be made of glass, silicon, paper, hydrogel, polymer monoliths, metal mesh, foam, ceramics, or other material. In some embodiments, the material may be naturally porous. In some embodiments, the material may have pores or wells etched into solid material. In some embodiments, the permeabilization reagents are flowed through a microfluidic chamber or channel over the porous membrane. In some embodiments, the flow controls the sample's access to the permeabilization reagents. In some embodiments, the porous membrane is a permeable hydrogel. For example, a hydrogel is permeable when permeabilization reagents and/or biological sample preparation reagents can pass through the hydrogel using diffusion. Any suitable permeabilization reagents and/or biological sample preparation reagents described herein can be used under conditions sufficient to release analytes (e.g., nucleic acid, protein, metabolites, lipids, etc.) from the biological sample. In some embodiments, a hydrogel is exposed to the biological sample on one side and permeabilization reagent on the other side. The permeabilization reagent diffuses through the permeable hydrogel and permeabilizes the biological sample on the other side of the hydrogel. In some embodiments, permeabilization reagents are flowed over the hydrogel at a variable flow rate (e.g., any flow rate that facilitates diffusion of the permeabilization reagent across the hydrogel). In some embodiments, the permeabilization reagents are flowed through a microfluidic chamber or channel over the hydrogel. Flowing permeabilization reagents across the hydrogel enables control of the concentration of reagents. In some embodiments, hydrogel chemistry and pore volume can be tuned to enhance permeabilization and limit diffusive analyte losses.

In some embodiments, a porous membrane is sandwiched between a spatially-barcoded array and the sample, wherein permeabilization solution is applied over the porous membrane. The permeabilization reagents diffuse through the pores of the membrane and into the biological sample. In some embodiments, the biological sample can be placed on a substrate (e.g., a glass slide). Biological analytes then diffuse through the porous membrane and into to the space containing the capture probes. In some embodiments, the porous membrane is modified to include capture probes. For example, the capture probes can be attached to a surface of the porous membrane using any of the methods described herein. In another example, the capture probes can be embedded in the porous membrane at any depth that allows interaction with a biological analyte. In some embodiments, the porous membrane is placed onto a biological sample in a configuration that allows interaction between the capture probes on the porous membrane and the biological analytes from the biological sample. For example, the capture probes are located on the side of the porous membrane that is proximal to the biological sample. In such cases, permeabilization reagents on the other side of the porous membrane diffuse through the porous membrane into the location containing the biological sample and the capture probes in order to facilitate permeabilization of the biological sample (e.g., also facilitating capture of the biological analytes by the capture probes). In some embodiments, the porous membrane is located between the sample and the capture probes. In some embodiments, the permeabilization reagents are flowed through a microfluidic chamber or channel over the porous membrane.

### (14) Other Reagents

Additional reagents can be added to a biological sample to perform various functions prior to analysis of the biological sample. In some embodiments, nuclease inhibitors such as DNase and RNase inactivating agents or protease inhibitors, and/or chelating agents such as EDTA, can be added to the biological sample. In other embodiments nucleases, such as DNase or RNAse, or proteases, such as pepsin or proteinase K, are added to the sample. In some embodiments, additional reagents may be dissolved in a solution or applied as a medium to the sample. In some embodiments, additional reagents (e.g., pepsin) may be dissolved in HCl prior to applying to the sample. For example, hematoxylin, from an H&E stain, can be optionally removed from the biological sample by washing in dilute HCl (0.001M to 0.1M) prior to further processing. In some embodiments, pepsin can be dissolved in dilute HCl (0.001M to 0.1M) prior to further processing. In some embodiments, biological samples can be washed additional times (e.g., 2, 3, 4, 5, or more times) in dilute HCl prior to incubation with a protease (e.g., pepsin), but after proteinase K treatment.

In some embodiments, the biological sample can be treated with one or more enzymes. For example, one or more endonucleases to fragment DNA, DNA polymerase enzymes, and dNTPs used to amplify nucleic acids can be added. Other enzymes that can also be added to the biological sample include, but are not limited to, polymerase, transposase, ligase, and DNAse, and RNAse.

In some embodiments, reverse transcriptase enzymes can be added to the sample, including enzymes with terminal transferase activity, primers, and template switch oligonucleotides (TSOs). Template switching can be used to increase the length of a cDNA, e.g., by appending a predefined nucleic acid sequence to the cDNA. In some embodiments, the appended nucleic acid sequence comprises one or more ribonucleotides.

In some embodiments, additional reagents can be added to improve the recovery of one or more target molecules (e.g., cDNA molecules, mRNA transcripts). For example, addition of a carrier RNA to an RNA sample workflow process can increase the yield of extracted RNA/DNA hybrids from the biological sample. In some embodiments, carrier molecules are useful when the concentration of input or target molecules is low as compared to remaining molecules. Generally, single target molecules cannot form a precipitate, and addition of the carrier molecules can help in forming a precipitate. Some target molecule recovery protocols use carrier RNA to prevent small amounts of target nucleic acids present in the sample from being irretrievably bound. In some embodiments, carrier RNA can be added immediately prior to a second strand synthesis step. In some embodiments, carrier RNA can be added immediately prior to a second strand cDNA synthesis on oligonucleotides released from an array. In some embodiments, carrier RNA can be added immediately prior to a post in vitro transcription clean-up step. In some embodiments, carrier RNA can be added prior to amplified RNA purification and quantification. In some embodiments, carrier RNA can be added before RNA quantification. In some embodiments, carrier RNA can be added immediately prior to both a second strand cDNA synthesis and a post in vitro transcription clean-up step.

### (15) Pre-processing for Capture Probe Interaction

In some embodiments, analytes in a biological sample can be pre-processed prior to interaction with a capture probe. For example, prior to interaction with capture probes, polymerization reactions catalyzed by a polymerase (e.g., DNA polymerase or reverse transcriptase) are performed in the biological sample. In some embodiments, a primer for the polymerization reaction includes a functional group that enhances hybridization with the capture probe. The capture probes can include appropriate capture domains to capture biological analytes of interest (e.g., poly(dT) sequence to capture poly(A) mRNA).

In some embodiments, biological analytes are pre-processed for library generation via next generation sequencing. For example, analytes can be pre-processed by addition of a modification (e.g., ligation of sequences that allow interaction with capture probes). In some embodiments, analytes (e.g., DNA or RNA) are fragmented using fragmentation techniques (e.g., using transposases and/or fragmentation buffers).

Fragmentation can be followed by a modification of the analyte. For example, a modification can be the addition through ligation of an adapter sequence that allows hybridization with the capture probe. In some embodiments, where the analyte of interest is RNA, poly(A) tailing is performed. Addition of a poly(A) tail to RNA that does not contain a poly(A) tail can facilitate hybridization with a capture probe that includes a capture domain with a functional amount of poly(dT) sequence.

In some embodiments, prior to interaction with capture probes, ligation reactions catalyzed by a ligase are performed in the biological sample. In some embodiments, ligation can be performed by chemical ligation. In some embodiments, the ligation can be performed using click chemistry as further described below. In some embodiments, the capture domain includes a DNA sequence that has complementarity to a RNA molecule, where the RNA molecule has complementarity to a second DNA sequence, and where the RNA-DNA sequence complementarity is used to ligate the second DNA sequence to the DNA sequence in the capture domain. In these embodiments, direct detection of RNA molecules is possible.

In some embodiments, prior to interaction with capture probes, target-specific reactions are performed in the biological sample. Examples of target specific reactions include, but are not limited to, ligation of target specific adaptors, probes and/or other oligonucleotides, target specific amplification using primers specific to one or more analytes, and target-specific detection using in situ hybridization, DNA microscopy, and/or antibody detection. In some embodiments, a capture probe includes capture domains targeted to target-specific products (e.g., amplification or ligation).

In some embodiments, a substrate comprises a capture probe attached to a capture area, wherein the capture probe comprises a spatial barcode and a capture domain, and wherein the capture domain binds directly or indirectly to a biological analyte from the biological sample or an intermediate agent indicative of the presence of the biological analyte.

### Capture Probes

Capture probes can be, e.g., attached to a surface, e.g., a solid support, a bead, or a coverslip. In some examples, capture probes are not attached to a surface. In some examples, capture probes can be encapsulated within, embedded within, or layered on a surface of a permeable composition (e.g., any of the substrates described herein). For example, capture probes can be encapsulated or disposed within a permeable bead (e.g., a gel bead). In some examples, capture probes can be encapsulated within, embedded within, or layered on a surface of a substrate (e.g., any of the exemplary substrates described herein, such as a hydrogel or a porous membrane).

In some examples, a cell or a tissue sample including a cell are contacted with capture probes attached to a substrate (e.g., a surface of a substrate), and the cell or tissue sample is permeabilized to allow analytes to be released from the cell and hybridize to the capture probes attached to the substrate. In some examples, analytes released from a cell can be actively directed to the capture probes attached to a substrate using a variety of methods alternative to diffusion, e.g., electrophoresis, chemical gradient, pressure gradient, fluid flow, or magnetic field.

In other examples, a capture probe can be directed to interact with a cell or a tissue sample using a variety of methods, e.g., inclusion of a lipid anchoring agent in the capture probe, inclusion of an agent that binds specifically to, or forms a covalent bond with a membrane protein in the capture probe, fluid flow, pressure gradient, chemical gradient, or magnetic field.

Non-limiting aspects of spatial analysis methodologies are described in WO 2011/127099, WO 2014/210233, WO 2014/210225, WO 2016/162309, WO 2018/091676, WO 2012/140224, WO 2014/060483, U.S. Pat. Nos. 10,002,316, 9,727,810, U.S. Patent Application Publication No. 2017/0016053, Rodriques et al., Science 363(6434):1463-1467, 2019; WO 2018/045186, Lee et al., Nat. Protoc. 10(3):442-458, 2015; WO 2016/007839, WO 2018/045181, WO 2014/163886, Trejo et al., PLoS ONE 14(2):e0212031, 2019, U.S. Patent Application Publication No. 2018/0245142, Chen et al., Science 348(6233):aaa6090, 2015, Gao et al., BMC Biol. 15:50, 2017, WO 2017/144338, WO 2018/107054, WO 2017/222453, WO 2019/068880, WO 2011/094669, U.S. Pat. Nos. 7,709,198, 8,604,182, 8,951,726, 9,783,841, 10,041,949, WO 2016/057552, WO 2017/147483, WO 2018/022809, WO 2016/166128, WO 2017/027367, WO 2017/027368, WO 2018/136856, WO 2019/075091, U.S. Pat. No. 10,059,990, WO 2018/057999, WO 2015/161173, and Gupta et al., Nature Biotechnol. 36:1197-1202, 2018, and can be used herein in any combination. Further non-limiting aspects of spatial analysis methodologies are described herein.

### Barcodes

A “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes.

Barcodes can have a variety of different formats. For example, barcodes can include non-random, semi-random, and/or random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell resolution (e.g., a barcode can be or can include a “spatial barcode”). In some embodiments, a barcode includes both a UMI and a spatial barcode. In some embodiments, a barcode includes two or more sub-barcodes that together function as a single barcode (e.g., a polynucleotide barcode). For example, a polynucleotide barcode can include two or more polynucleotide sequences (e.g., sub-barcodes) that may be separated by one or more non-barcode sequences.

### RNA-Templated Ligation

In some embodiments of methods provided here, RNA-templated ligation is used to interrogate spatial gene expression in a biological sample (e.g., an FFPE tissue section). RNA-templated ligation enables sensitive measurement of specific nucleic acid analytes of interest that otherwise might be analyzed less sensitively with a whole transcriptomic approach. It provides advantages of compatibility with common histochemical stains and suitability for analysis of decade-old materials (e.g., FFPE samples) and exceedingly small microdissected tissue fragments.

In some aspects, the steps of RNA-templated ligation include: (1) hybridization of pairs of probes (e.g., DNA probes) to RNA (e.g., formalin fixed RNA) within a tissue section; (2) ligation of adjacently annealed probe pairs in situ; (3) RNase H treatment that (i) releases RNA-templated ligation products from the tissue (e.g., into solution) for downstream analysis and (ii) destroys unwanted DNA-templated ligation products; and optionally, (4) amplification of RNA-templated ligation products (e.g., by multiplex PCR).

In some aspects, disclosed herein are methods of direct detection of RNA target-DNA probe duplexes without first converting RNA to cDNA by reverse transcription. In some aspects, RNA templated ligation includes a ligase that ligates adjacently positioned single stranded DNA that have hybridized to a RNA target nucleic acid, such as an enzyme or functional variant thereof from a Chlorella virus (e.g., PBCV-1 DNA ligase, ATCV-1 DNA ligase). In some aspects, RNA-templated ligation can include a DNA ligase.

In some aspects, RNA-templated ligation is used for detection of RNA, determination of RNA sequence identity, and/or expression monitoring and transcript analysis. In some aspects, RNA-templated ligation allows for detection of a particular change in a nucleic acid (e.g., a mutation or single nucleotide polymorphism (SNP)), detection or expression of a particular nucleic acid, or detection or expression of a particular set of nucleic acids (e.g., in a similar cellular pathway or expressed in a particular pathology). In some embodiments, the methods that include RNA-templated ligation are used to analyze nucleic acids, e.g., by genotyping, quantitation of DNA copy number or RNA transcripts, localization of particular transcripts within samples, and the like. In some aspects, systems and methods provided herein that include RNA-templated ligation identify single nucleotide polymorphisms (SNPs). In some aspects, such systems and methods identify mutations.

In some aspects, disclosed herein are methods of detecting RNA expression that include bringing into contact a first probe, a second probe, and ligase. In some embodiments, the first probe and the second probe are designed to hybridize to a target sequence such that the 5′ end of the first probe and the 3′ end of the second probe are adjacent and can be ligated, wherein at least the 5′-terminal nucleotide of the first probe and at least the 31-terminal nucleotide of the second probe are deoxyribonucleotides (DNA), and wherein the target sequence includes (e.g., is composed of) ribonucleotides (RNA). After hybridization, a ligase ligates the first probe and the second probe if the target sequence is present in the target sample, but does not ligate the first probe and the second probe if the target sequence is not present in the target sample. The presence or absence of the target sequence in the biological sample can be determined by determining whether or not the first and second probes were ligated in the presence of ligase. Any of a variety of methods can be used to determine whether or not the first and second probes were ligated in the presence of ligase, including but not limited to, sequencing the ligated product, hybridizing the ligated product with a detection probe that hybridizes only when the first and second probes were ligated in the presence of ligase, restriction enzyme analysis, and other methods known in the art.

In some aspects, two or more RNA analytes are analyzed using methods that include RNA-templated ligation. In some aspects, when two or more analytes are analyzed, a first and second probe that is specific for (e.g., specifically hybridizes to) each RNA analyte are used.

In some aspects, the first probe is a DNA probe. In some aspects, the first probe is a chimeric DNA/RNA probe. In some aspects, the second probe is a DNA probe. In some aspects, the second probe is a chimeric DNA/RNA probe.

In some aspects, a ligation product forms a DNA:RNA hybrid with the RNA target of interest, wherein subsequent treatment with RNase H releases RNA-templated ligation products from the RNA target for capture by the capture domain of a capture probe located in proximity to the hybridization and ligation events.

In some embodiments of methods provided herein, a biological sample is a fresh frozen tissue sample, and the capture domain binds to an mRNA released from the cell; or wherein the biological sample is a formalin-fixed paraffin-embedded (FFPE) tissue sample, and the capture domain binds to a ligation product that is a proxy for a target mRNA in the biological sample.

### Inserts

In some embodiments of methods provided herein, a porous insert suppresses convection flow and/or suppresses displacement of nucleic acids or nucleic acid proxies (e.g., an intermediate agent such as a ligation product) away from a location in a biological sample.

A porous insert used in practicing methods of the present disclosure to mitigate TML may be composed of one or more materials. In some embodiments of methods provided herein, a porous insert comprises a hydrogel, a foam, a metal mesh, a porous ceramic, or a combination thereof. Non-limiting examples of a hydrogel include poly(ethyleneglycol diacrylate) (PEGDA), poly(hydroxyethyl methacrylate) (pHEMA), agarose, alginate, poly(acrylamide), methylcellulose, collagen, gelatin, poly(acrylic acid), poly(ethyleneglycol dimethacrylate), poly(vinyl pyrrolidone), carboxymethyl cellulose, chitosan, poly(vinyl alcohol), chitin, and carrageenan. Non-limiting examples of a foam include polyethylene (PE), polyurethane (PU), polystyrene (PS), polyvinyl (PVC), rubber foam, and thermoplastic elastomer foam. Non-limiting examples of a metal mesh include aluminum, brass, bronze, copper, and steel. Non-limiting examples of a porous ceramic include silicate, diatomite, carbon, corundum, silicon carbide, and ocordierite.

In some embodiments of methods provided herein, a porous insert comprises polyethylene, polypropylene, polyurethane foam, poly(ethylene glycol) diacrylate (PEGDA), hydrogel, metal, ceramics, or a combination thereof. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% PEGDA. In some embodiments, a porous insert comprises between about 100% PEGDA. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyethylene. In some embodiments, a porous insert comprises between about 100% polyethylene. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polypropylene. In some embodiments, a porous insert comprises between about 100% polypropylene. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyurethane foam. In some embodiments, a porous insert comprises between about 100% polyurethane foam. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyurethane foam. In some embodiments, a porous insert comprises between about 100% polyurethane foam.

In some embodiments of methods provided herein, a porous insert contacts a biological sample. In some aspects of methods provided herein, a porous insert is immediately adjacent to the biological sample. For example, in such embodiments, a porous insert may be separated from a biological sample by a thin layer of fluid. A thin layer of fluid may have a depth in a range from about 1-50 μm. A porous insert may be secured in place by making contact with a cassette or gasket in which the porous insert is disposed. In some embodiments of methods provided herein, a porous insert is separated from the biological sample by a spacer. A spacer can have a thickness in a range from about from about 1 μm to about 50 μm, about 2 μm to about 40 μm, about 3 μm to about 35 μm, about 4 μm to about 30 μm, about 5 μm to about 25 μm, or about 5 μm to about 20 μm. In some embodiments, a spacer can have a thickness of at least 1 μm, at least 2 μm, at least 5 μm, at least 10 μm, at least 20 μm, or at least 50 μm or more.

In some embodiments, provided herein are methods for correlating a location of a nucleic acid in a biological sample. In some embodiments, a method for correlating a location of a nucleic acid in a biological sample comprises (1) providing a substrate comprising a capture area and a capture probe attached to the capture area; (2) inserting a porous insert in the opening of the chamber; (3) capturing a nucleic acid from the biological sample or an intermediate agent indicative of the presence of the nucleic acid in the biological sample using the capture probe; and (4) determining the sequence of the spatial barcode of the capture probe, or a complement thereof, and all or a portion of the sequence of the nucleic acid, or a complement thereof, and using the sequences to correlate the location of the nucleic acid to its location in the biological sample. In some embodiments, a method for correlating a location of a nucleic acid in a biological sample comprises (1) providing a substrate comprising a capture area and a capture probe attached to the capture area, wherein the capture probe comprises a spatial barcode and a capture domain, a biological sample disposed on the capture area, a buffer disposed on the biological sample, and a gasket disposed on the substrate, wherein the gasket provides a chamber that comprises an opening encompassing the lateral dimension of the capture area and includes a height above the biological sample and buffer; (2) inserting a porous insert in the opening of the chamber wherein the porous insert is in contact with the buffer and/or the biological sample, and wherein the porous insert limits free flow space in the chamber, thereby reducing fluid convection above the biological sample; (3) capturing a nucleic acid from the biological sample or an intermediate agent indicative of the presence of the nucleic acid in the biological sample using the capture probe; and (4) determining the sequence of the spatial barcode of the capture probe, or a complement thereof, and all or a portion of the sequence of the nucleic acid, or a complement thereof, and using the sequences to correlate the location of the nucleic acid to its location in the biological sample. In some embodiments, a method for correlating a location of a nucleic acid in a biological sample comprises (1) providing a substrate comprising a capture area and a capture probe attached to the capture area, wherein the capture probe comprises a spatial barcode and a capture domain, a biological sample disposed on the capture area, and a gasket disposed on the substrate, wherein the gasket provides a chamber that comprises an opening encompassing the lateral dimension of the capture area and includes a height above the biological sample; (2) inserting a porous insert in the opening of the chamber wherein the porous insert is in contact with and/or adjacent to the biological sample, and wherein the porous insert limits free flow space in the chamber, thereby reducing fluid convection above the biological sample; (3) disposing a buffer above the insert so that the buffer contacts the insert and the biological sample; (4) capturing a nucleic acid from the biological sample or an intermediate agent indicative of the presence of the nucleic acid in the biological sample using the capture probe; and (5) determining the sequence of the spatial barcode of the capture probe, or a complement thereof, and all or a portion of the sequence of the nucleic acid, or a complement thereof, and using the sequences to correlate the location of the nucleic acid to its location in the biological sample. In some embodiments, a method for correlating a location of a nucleic acid in a biological sample comprises (1) providing a substrate comprising a capture area and a capture probe attached to the capture area, wherein the capture probe comprises a spatial barcode and a capture domain, a biological sample disposed on the capture area, and a gasket disposed on the substrate, wherein the gasket provides a chamber that comprises an opening encompassing the lateral dimension of the capture area and includes a height above the biological sample; (2) inserting a porous insert in the opening of the chamber wherein the porous insert is in contact with and/or adjacent to the biological sample, and wherein the porous insert limits free flow space in the chamber, thereby reducing fluid convection above the biological sample; (3) disposing a buffer in a space surrounding the insert so that the buffer contacts the insert and the biological sample; (4) capturing a nucleic acid from the biological sample or an intermediate agent indicative of the presence of the nucleic acid in the biological sample using the capture probe; and (5) determining the sequence of the spatial barcode of the capture probe, or a complement thereof, and all or a portion of the sequence of the nucleic acid, or a complement thereof, and using the sequences to correlate the location of the nucleic acid to its location in the biological sample.

In some embodiments, provided herein are methods for identifying a location of a nucleic acid in a biological sample. In some embodiments, a method for identifying a location of a nucleic acid in a biological sample comprises (1) providing a substrate comprising a capture area and a capture probe attached to the capture area; (2) inserting a porous insert in the gasketed chamber; (3) capturing a nucleic acid or an intermediate agent indicative of the presence of the nucleic acid in the biological sample by the capture probe; and (4) determining the sequence of the spatial barcode of the capture probe, or a complement thereof, and all or a portion of the sequence of the nucleic acid from the biological sample, or a complement thereof, and using the sequences to identify the location of the nucleic acid in the biological sample. In some embodiments, a method for identifying a location of a nucleic acid in a biological sample comprises (1) providing a substrate comprising a capture area and a capture probe attached to the capture area, wherein the capture probe comprises a spatial barcode and a capture domain, a biological sample disposed on the capture area, and a gasket disposed on the substrate, wherein the gasket comprises an opening encompassing the lateral dimension of the capture area and a height above the biological sample; (2) inserting a porous insert in the opening, wherein the porous insert is pre-filled with a buffer and is disposed on the biological sample, wherein the porous insert limits free flow space in the chamber, thereby reducing fluid convection above the biological sample; (3) capturing a nucleic acid or an intermediate agent indicative of the presence of the nucleic acid in the biological sample by the capture probe; and (4) determining the sequence of the spatial barcode of the capture probe, or a complement thereof, and all or a portion of the sequence of the nucleic acid from the biological sample, or a complement thereof, and using the sequences to identify the location of the nucleic acid in the biological sample.

### V. Insert Compositions

Provided herein are compositions useful to reduce the mislocalization of a target analyte (e.g., mRNA transcript) during spatial analysis as described above. In some embodiments, a composition of the present disclosure comprises: (1) a substrate comprising a capture area, a biological sample disposed on the capture area, and a buffer disposed on the biological sample; (2) a gasket disposed on the substrate, wherein the gasket comprises an opening encompassing the lateral dimension of the capture area; and (3) a porous insert inserted in the opening and in contact with the buffer and/or the biological sample. In some embodiments, the porous insert defines a height of the gasketed area and limits free flow space in the gasketed area. In some embodiments, the substrate comprises a capture probe attached to the capture area, wherein the capture probe comprises a spatial barcode and a capture domain, and wherein the capture domain binds directly or indirectly to a nucleic acid from the biological sample or an intermediate agent indicative of the presence of the nucleic acid.

In some embodiments, a provided composition reduces fluid convection above a biological sample, relative to a composition that does not comprise an insert. In some embodiments, a provided composition reduces upward movement of a nucleic acid, or an intermediate agent released from a biological sample, relative to a composition that does not comprise an insert. In some embodiments, a provided composition reduces lateral movement of a nucleic acid, or an intermediate agent released from a biological sample, relative to a composition that does not comprise an insert. In some embodiments, a composition (i) reduces fluid convection above the biological sample, (ii) reduces upward movement of a nucleic acid or an intermediate agent released from the biological sample; and/or (iv) reduces lateral movement of a biological analyte or an intermediate agent released from the biological sample.

In some embodiments, a composition comprises a capture probe comprising a unique molecular identifier (UMI).

In some embodiments of compositions provided herein, a porous insert suppresses convection flow and/or suppresses lateral movement of a biological analyte away from a biological sample.

A porous insert may be composed of one or more materials. In some embodiments of compositions provided herein, a porous insert comprises a hydrogel, a foam, a metal mesh, a porous ceramic, or a combination thereof. Non-limiting examples of the hydrogel include poly(ethyleneglycol diacrylate) (PEGDA), poly(hydroxyethyl methacrylate) (pHEMA), agarose, alginate, poly(acrylamide), methylcellulose, collagen, gelatin, poly(acrylic acid), poly(ethyleneglycol dimethacrylate), poly(vinyl pyrrolidone), carboxymethyl cellulose, chitosan, poly(vinyl alcohol), chitin, and carrageenan. Non-limiting examples of the foam include polyethylene (PE), polyurethane (PU), polystyrene (PS), polyvinyl (PVC), rubber foam, and thermoplastic elastomer foam. Non-limiting examples of the metal mesh include aluminum, brass, bronze, copper, and steel. Non-limiting examples of the porous ceramic include silicate, diatomite, carbon, corundum, silicon carbide, and ocordierite.

In some embodiments of compositions provided herein, a porous insert comprises polyethylene, polypropylene, polyurethane foam, poly(ethylene glycol) diacrylate (PEGDA), hydrogel, metal, ceramics, or a combination thereof. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% PEGDA by volume. In some embodiments, a porous insert comprises about 100% PEGDA. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyethylene by volume. In some embodiments, a porous insert comprises about 100% polyethylene. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polypropylene by volume. In some embodiments, a porous insert comprises about 100% polypropylene. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyurethane foam by volume. In some embodiments, a porous insert comprises about 100% polyurethane foam.

In some embodiments of compositions provided herein, a porous insert contacts a biological sample. In some aspects of methods provided herein, a porous insert is immediately adjacent to the biological sample. For example, in such embodiments, a porous insert may be separated from a biological sample by a thin layer of fluid. A thin layer of fluid may have a depth in a range from about 1-50 μm. A porous insert may be secured in place by making contact with a cassette or gasket in which the porous insert is disposed. In some embodiments of compositions provided herein, a porous insert is separated from the biological sample by a spacer. A spacer may comprise one or more materials, including but not limited to plastics such as polyethylene terephthalate (PET) and photoresist. A spacer can comprise the same material(s) as an insert of the disclosure, itself. A spacer can have a thickness in a range from about from about 1 μm to about 50 μm, about 2 μm to about 40 μm, about 3 μm to about 35 μm, about 4 μm to about 30 μm, about 5 μm to about 25 μm, or about 5 μm to about 20 μm. In some embodiments, a spacer can have a thickness of at least 1 μm, at least 2 μm, at least 5 μm, at least 10 μm, at least 20 μm, or at least 50 μm or more.

In some embodiments of compositions provided herein, a porous insert comprises a plurality of pores. In some embodiments of compositions provided herein, a porous insert comprises a plurality of pores, each pore having a diameter in a range from about 10 nm to about 1000 μm, about 50 nm to about 500 μm, about 100 nm to about 500 μm, about 500 nm to about 500 μm, about 1 μm to about 100 μm, or about 10 μm to about 100 μm. In some embodiments of compositions provided herein, a porous insert comprises a plurality of pores, each pore having a diameter of about 10 μm, about 20 μm, about 30 μm, about 40 μm, about 50 μm, about 60 μm, about 70 μm, about 80 μm, about 90 μm, or about 100 μm.

In some embodiments of compositions provided herein, a porous insert comprises a hydrophilic material, and has an overall high hydrophilicity. In some embodiments, a porous insert has a contact angle between 0° and about 80°, between 0° and about 70°, between 0° and about 60°, between 0° and about 50°, between 0° and about 40°, between 0° and about 30°, between 0° and about 20°, or between 0° and about 10°. In some preferred embodiments, a porous insert has a contact angle between 0° and about 60°.

In some embodiments of compositions provided herein, a porous insert has a compressibility in a range from about 1×10⁻⁵ m²/N to about 1×10⁻⁸ m²/N. In some preferred embodiments, a porous insert has a compressibility in a range from about 1×10⁻⁶ m²/N to about 1×10⁻⁷ m²/N.

In some embodiments of compositions provided herein, a porous insert is thermally stable. In some embodiments of compositions provided herein, a porous insert is thermally stable at a temperature in a range from about 15° C. to about 60° C. In some embodiments of compositions provided herein, a porous insert is thermally stable at about 37° C.

In some embodiments of compositions provided herein, a porous insert can comprise a supporting material. A supporting material may be useful for handling a porous insert, for example, when positioning the porous insert into a gasket, or removing a porous insert from a gasket. For example, a holder or cap may be placed on top of a porous insert to enable insert placement, manipulation, or removal. A holder or cap can be made of plastic, metal, a combination thereof, or other materials.

### VI. Kits

This disclosure provides kits, including kits useful for performing methods described herein. In some embodiments, a kit may include, without limitation, (1) a substrate comprising a capture area for receiving a biological sample; (2) a gasket configured to be disposed on the substrate, wherein when disposed on the substrate the gasket comprises an opening encompassing the lateral dimension of the capture area; and (3) a porous insert configured to be inserted in the opening. In some embodiments of kits provided herein, a porous insert, when inserted in the opening, defines a height of the gasketed area and limits free flow space in the gasketed area, thereby (i) reducing fluid convection above the biological sample, (ii) reducing upward movement of a biological analyte or an intermediate agent released from the biological sample.

In some embodiments of kits provided herein, a substrate comprises a capture probe attached to the capture area, wherein the capture probe comprises a spatial barcode and a capture domain. In some embodiments of kits provided herein, a capture probe comprises a unique molecular identifier (UMI). A porous insert of a kit provided herein may be prefilled with a buffer (e.g., buffer solution, e.g., permeabilization solution) for disposing on a biological sample. A kit may include a buffer comprising a permeabilization enzyme.

In some embodiments of kits provided herein, a porous insert suppresses convection flow and/or suppresses lateral movement of a biological analyte away from a biological sample.

In some embodiments of kits provided herein, a porous insert comprises a hydrogel, a foam, a metal mesh, a porous ceramic, or a combination thereof. Non-limiting examples of the hydrogel include poly(ethyleneglycol diacrylate) (PEGDA), poly(hydroxyethyl methacrylate) (pHEMA), agarose, alginate, poly(acrylamide), methylcellulose, collagen, gelatin, poly(acrylic acid), poly(ethyleneglycol dimethacrylate), poly(vinyl pyrrolidone), carboxymethyl cellulose, chitosan, poly(vinyl alcohol), chitin, and carrageenan. Non-limiting examples of the foam include polyethylene (PE), polyurethane (PU), polystyrene (PS), polyvinyl (PVC), rubber foam, and thermoplastic elastomer foam. Non-limiting examples of the metal mesh include aluminum, brass, bronze, copper, and steel. Non-limiting examples of the porous ceramic include silicate, diatomite, carbon, corundum, silicon carbide, and ocordierite.

In some embodiments of kits provided herein, a porous insert comprises polyethylene, polypropylene, polyurethane foam, poly(ethylene glycol) diacrylate (PEGDA), hydrogel, metal, ceramics, or a combination thereof. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% PEGDA. In some embodiments, a porous insert comprises between about 100% PEGDA. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyethylene. In some embodiments, a porous insert comprises between about 100% polyethylene. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polypropylene. In some embodiments, a porous insert comprises between about 100% polypropylene. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyurethane foam. In some embodiments, a porous insert comprises between about 100% polyurethane foam. In some embodiments, a porous insert comprises between about 0.1% to about 99%, about 1% to about 99%, about 5% to about 90%, about 10% to about 80%, about 20% to about 70%, about 30% to about 60%, about 30% to about 50%, about 35% to about 45%, or about 40% polyurethane foam. In some embodiments, a porous insert comprises between about 100% polyurethane foam.

In some embodiments of kits provided herein, a porous insert contacts a biological sample. In some aspects of methods provided herein, a porous insert is immediately adjacent to the biological sample. For example, in such embodiments, a porous insert may be separated from a biological sample by a thin layer of fluid. A thin layer of fluid may have a depth in a range from about 1-50 μm. A porous insert may be secured in place by making contact with a cassette or gasket in which the porous insert is disposed. In some embodiments of kits provided herein, a porous insert is separated from the biological sample by a spacer. A spacer may comprise one or more materials, including but not limited to plastics such as polyethylene terephthalate (PET) and photoresist. A spacer can comprise the same material(s) as an insert of the disclosure, itself. A spacer can have a thickness in a range from about from about 1 μm to about 50 μm, about 2 μm to about 40 μm, about 3 μm to about 35 μm, about 4 μm to about 30 μm, about 5 μm to about 25 μm, or about 5 μm to about 20 μm. In some embodiments, a spacer can have a thickness of at least 1 μm, at least 2 μm, at least 5 μm, at least 10 μm, at least 20 μm, or at least 50 μm or more.

In some embodiments of kits provided herein, a porous insert comprises a plurality of pores. In some embodiments of kits provided herein, a porous insert comprises a plurality of pores, each pore having a diameter in a range from about 10 nm to about 1000 μm, about 50 nm to about 500 μm, about 100 nm to about 500 μm, about 500 nm to about 500 μm, about 1 μm to about 100 μm, or about 10 μm to about 100 μm. In some embodiments of kits provided herein, a porous insert comprises a plurality of pores, each pore having a diameter of about 10 μm, about 20 μm, about 30 μm, about 40 μm, about 50 μm, about 60 μm, about 70 μm, about 80 μm, about 90 μm, or about 100 μm.

In some embodiments of compositions provided herein, a porous insert comprises a hydrophilic material, and has an overall high hydrophilicity. In some embodiments, a porous insert has a contact angle between 0° and 80°, between 0° and 70°, between 0° and 60°, between 0° and 50°, between 0° and 40°, between 0° and 30°, between 0° and 20°, or between 0° and 10°.

In some embodiments of kits provided herein, a porous insert has a compressibility in a range from about 1×10⁻⁵ m²/N to about 1×10⁻⁸ m²/N. In some preferred embodiments, a porous insert has a compressibility in a range from about 1×10⁻⁶ m²/N to about 1×10⁻⁷ m²/N.

In some embodiments of kits provided herein, a porous insert is thermally stable. In some embodiments of kits provided herein, a porous insert is thermally stable at a temperature in a range from about 15° C. to about 60° C. In some embodiments of kits provided herein, a porous insert is thermally stable at about 37° C.

In some embodiments of kits provided herein, a porous insert can comprise a supporting material. A supporting material may be useful for handling a porous insert, for example, when positioning the porous insert into a gasket, or removing a porous insert from a gasket. For example, a holder or cap may be placed on top of a porous insert to enable insert placement, manipulation, or removal. A holder or cap can be made of plastic, metal, a combination thereof, or other materials.

### EXAMPLES

### Example 1. Sources of Transcript Mislocalization (TML)

Transcript mislocalization (TML) can occur during spatial analysis of target analytes for a variety of reasons. Upon permeabilization of a tissue sample, fluid convection may result in the target analyte moving away from a spatially corresponding region on a capture area or surface. This may result in inaccurate assessment of the native location of the target analyte in tissue, thereby decreasing spatial expression resolution. Fluid convection can occur due to temperature gradients in the buffer through which a target analyte flows before contacting a capture area or surface. Marangoni flow (i.e., in a fluid with a surface temperature gradient, the flow of fluid away from low-surface tension areas), is a source of TML. Marangoni flow can be caused by temperature gradients, as temperature is inversely related to surface tension.

TML is more likely to occur when the solution volume is high. In other words, the larger volume of a fluid, the greater the propensity for convection to occur. Fluid convection may result in the lateral movement of an mRNA transcript, resulting in inaccurate determination of the location from which the transcript exited a tissue sample.

Other sources of TML include, without limitation, fluid leaks in the apparatus, non-uniform tissue thickness, apparatus movement or tilting, air bubbles, and gaps between the tissue and the slide.

### Example 2. Methods for Reducing TML

TML may be reduced by countering any of the numerous TML sources, including but not limited to those outlined in Example 1.

One method is to reduce fluid convection around a tissue sample to be analyzed. This can be accomplished by decreasing the height (and, in effect, volume), of a buffer solution within which a biological sample is placed for processing and spatial analysis. Reducing convection may also be accomplished by reducing the temperature gradient within a buffer containing a biological sample to be analyzed. For example, utilizing techniques that ensure a uniform temperature throughout the buffer volume may greatly reduce convection. A heated lid may be used to stabilize the temperature of the top of the buffer, which has a tendency to be of a lower temperature than buffer that is closer to the bottom of the analyte capture system. A third way convection can be reduced is by increasing the viscosity of a buffer solution within which a biological sample is placed for processing and spatial analysis.

### Example 3. TML Reduction with a Porous Insert Structure

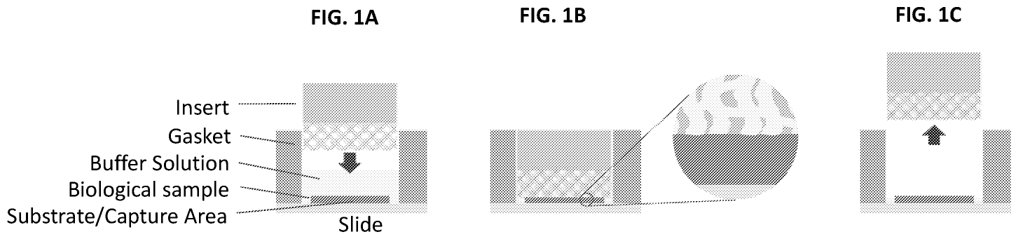

The present Example illustrates the use of a porous insert structure to reduce TML. As shown in FIG. 1A-1C, a gasket is disposed around an arrayed area on the substrate thereby creating an assay chamber, a biological sample is placed on the substrate comprising a capture area within the gasketed area (gasketed area further comprising fiducial marks surrounding the arrayed area). A buffer, such as a permeabilization solution, may be disposed on the biological sample. A porous insert can be inserted into the gasket chamber (FIG. 1A) such that the insert is immersed partially or fully in the buffer (FIG. 1B). The porous insert can be placed in direct contact or in close proximity to the biological sample (FIG. 1B). The surface area of a bottom face of the porous insert can be larger than the surface area of the biological sample, such that the porous insert surface completely covers the biological sample. Additional buffer solutions, such as a permeabilization solution, may be added in order to ensure that the biological sample remains wet. Due to the porous nature of the insert, a buffer solution can wick into the insert through capillary action (FIG. 1B). Following the completion of an incubation period (such as, for example, tissue permeabilization), the insert may be removed (FIG. 1C). Downstream processing steps can then be performed. The presence of the insert limits convection flow in the cassette or gasket, leading to a reduction in TML.

### Example 4. Porous Insert Design Considerations

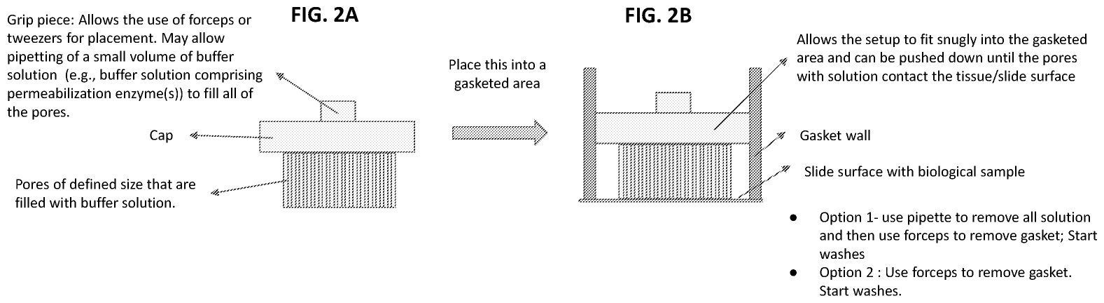

The present Example illustrates various design considerations for porous inserts of the present disclosure. As shown in FIG. 2A-2B, a porous insert may include a cap with a grip piece that allows for the use of forceps or other device for placement of the insert into a gasket chamber. The porous insert may include a cap that allows the addition of a volume of buffer solution (e.g., a permeabilization solution containing a permeabilization enzyme), via pipette or other means, in order to fill the pores of the porous insert with the buffer (FIG. 2A). The insert can be placed into a gasket chamber using forceps or other means, such that the insert fits snugly into the gasketed area (FIG. 2B). The insert can be manually pushed downward until the pores of the insert make contact with or become close in proximity to the biological sample.

Following sample incubation (e.g., tissue permeabilization with a permeabilization enzyme), the buffer solution can be removed while the insert remains in place. This may be accomplished by pipette or other means. Alternatively, the insert can be removed without pre-removal of the buffer solution. Washing of the biological sample can begin immediately following insert removal (FIG. 2B).

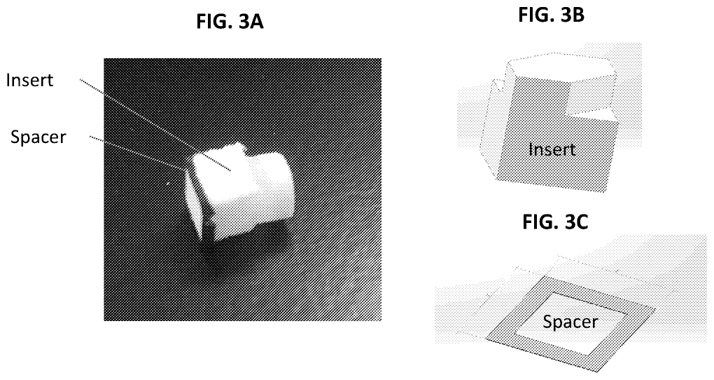

In certain circumstances, it may be desirable for the insert to be separated from (i.e., not directly in contact with) the biological sample. As shown in FIG. 3A-3C, a spacer may be placed on the bottom of an insert such that, when positioned in a gasket chamber above a biological sample, the insert does not contact the biological sample.

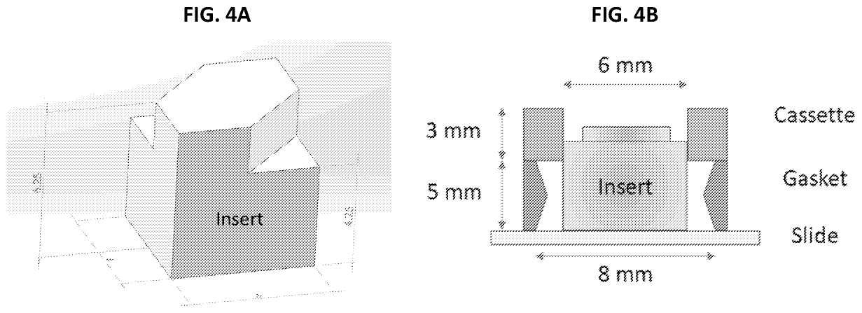

FIG. 4A illustrates an exemplary insert shape suitable for in insert that can be loaded into a gasket chamber. The insert can be held in place by the cassette frame in order to minimize movement during processing of a biological sample (FIG. 4B).

An insert may be composed of various materials including but not limited to polyethylene, polypropylene, polyurethane foam, hydrogel, metal mesh, porous ceramic, and combinations thereof. Insert pore size can range from nanometers to micrometers.

### Example 5. Effect of Insert on Transcript Detection Sensitivity

The present Example illustrates that the inclusion of a porous insert structure as described supra when performing a mouse brain tissue spatial assay does not result in a reduction in transcript detection sensitivity. Briefly, mouse brain tissue samples were isolated and sectioned to a thickness of 10 μm. Tissue sections were placed on an arrayed areas on a substrate and a gasket we affixed to the substrate thereby creating reaction chambers for the tissue sections on the arrayed areas for processing following the Visium protocol (Visium Spatial Gene Expression Reagent Kits-User Guide, 10× Genomics, (Rev F, Jan. 24, 2022), support. 10xgenomics.com/spatial-gene-expression/library-prep/doc/user-guide-visium-spatial-gene-expression-reagent-kits-user-guide). Transcript analysis was performed using the SpaceRanger pipeline and Loupe Browser visualization tools (10× Genomics).

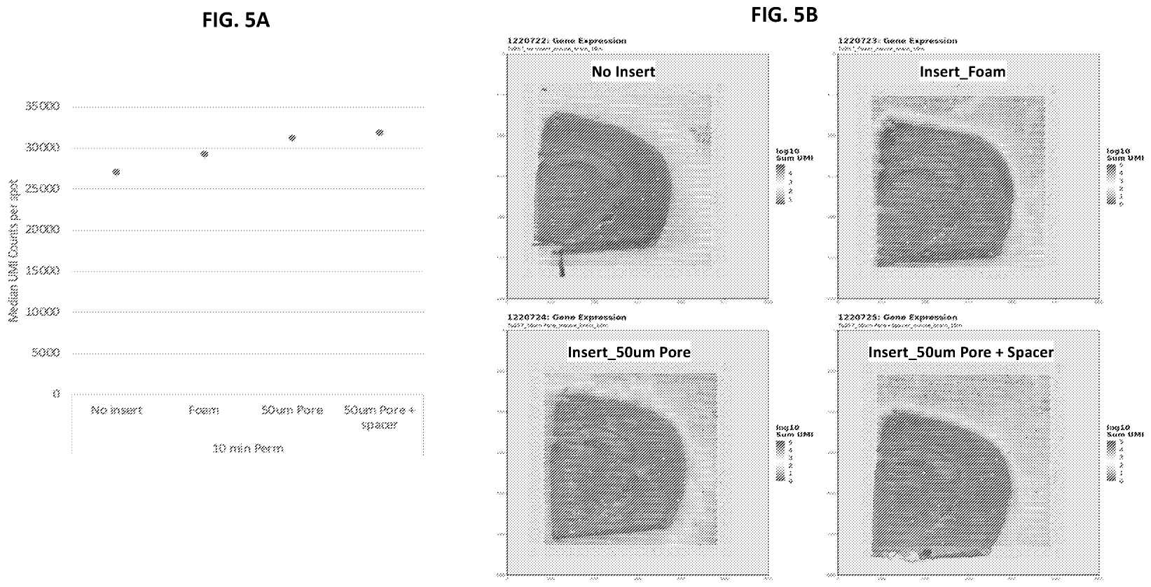

Four different insert conditions were evaluated: 1) no insert; 2) foam insert; 3) 50 μm pore insert; and 4) 50 μm pore insert plus a spacer. The presence of the insert did not result in a reduction in sensitivity, as measured by median UMI counts per assay spot or feature (FIG. 5A). Moreover, fewer UMI counts were detected in non-tissue areas when an Insert was used (smaller yellow halo (fewer UMI counts)), when compared to the No Insert control (larger yellow halo (increased UMI counts)), demonstrating that TML was reduced in each condition relative to the “No insert” condition (FIG. 5B), in accordance with sequencing data as presented in FIG. 5A. The use of the spacer increased the number of captured transcripts captured by decreasing TML as evidenced by the increase of UMI counts from “No insert” compared to “Insert” conditions, as such TMIL can be mitigated and spatial resolution of gene expression enhanced when using a porous insert of the present invention.

1. A method for reducing fluid convection in a chamber, comprising:
providing a substrate comprising a capture area, a biological sample disposed on the capture area, a buffer disposed on the biological sample, and a gasket disposed on the substrate surrounding the capture area, wherein the gasket provides a chamber comprising the lateral dimension of the capture area and includes a height above the capture area and biological sample; and
inserting a porous insert positioned in the chamber and in contact with the buffer and/or the biological sample, wherein the porous insert limits free flow space in the chamber, thereby reducing fluid convection in the chamber.

2. (canceled)

3. The method of claim 1, wherein the substrate comprises a capture probe attached to the capture area, wherein the capture probe comprises a spatial barcode and a capture domain, and wherein the capture domain hybridizes directly or indirectly to a biological analyte from the biological sample or an intermediate agent indicative of the presence of the biological analyte.

4. The method of claim 3, wherein the biological sample is a fresh frozen tissue sample, and the capture domain hybridizes to an mRNA released from the cell; or wherein the biological sample is a formalin-fixed paraffin-embedded (FFPE) tissue sample, and the capture domain hybridizes to a ligation product that is a proxy for a target mRNA in the biological sample.

5. The method of claim 1, wherein the porous insert comprises a hydrogel, a foam, a metal mesh, a porous ceramic, or a combination thereof.

6. The method of claim 5, wherein the hydrogel comprises poly(ethyleneglycol diacrylate), poly(hydroxyethyl methacrylate), agarose, alginate, poly(acrylamide), methylcellulose, collagen, gelatin, poly(acrylic acid), poly(ethyleneglycol dimethacrylate), poly(vinyl pyrrolidone), carboxymethyl cellulose, chitosan, poly(vinyl alcohol), chitin, carrageenan, or a combination thereof.

7. The method of claim 6, wherein the poly(ethyleneglycol diacrylate) is about 30% to 50% of the hydrogel.

8. The method of claim 5, wherein the foam comprises polyethylene, polyurethane, polystyrene, polyvinyl, rubber foam, thermoplastic elastomer foam, or a combination thereof.

9. The method of claim 5, wherein the metal mesh comprises aluminum, brass, bronze, copper, steel, or a combination thereof.

10. The method of claim 5, wherein the porous ceramic comprises silicate, diatomite, carbon, corundum, silicon carbide, ocordierite, or a combination thereof.

11. The method of claim 1, wherein the porous insert contacts the biological sample.

12. (canceled)

13. The method of claim 1, wherein the porous insert is separated from the biological sample by a spacer.

14. The method of claim 13, wherein the spacer has a thickness in a range from about 5 μm to about 20 μm.

15. The method of claim 1, wherein the buffer comprises a permeabilization enzyme.

16. A method for correlating a location of a biological analyte in a biological sample, comprising:
providing a substrate comprising a capture area and a capture probe attached to the capture area, wherein the capture probe comprises a spatial barcode and a capture domain, a biological sample disposed on the capture area, a buffer disposed on the biological sample, and a gasket disposed on the substrate, wherein the gasket surrounds the capture area and provides a chamber that comprises an opening encompassing the lateral dimension of the capture area and includes a height above the biological sample and buffer;
inserting a porous insert in the opening of the chamber wherein the porous insert is in contact with the buffer and/or the biological sample, and wherein the porous insert limits free flow space in the chamber, thereby reducing fluid convection above the biological sample;
capturing a biological analyte from the biological sample or an intermediate agent indicative of the presence of the biological analyte in the biological sample using the capture probe; and
determining the sequence of the spatial barcode of the capture probe or a complement thereof, and the sequence of the biological analyte or a complement thereof, and using the sequences to correlate the location of the biological analyte to its location in the biological sample.

17.-39. (canceled)

40. A composition comprising:
a substrate comprising a capture area, a biological sample disposed on the capture area, and a buffer disposed on the biological sample;
a gasket disposed on the substrate, wherein the gasket generates a chamber encompassing the lateral dimension of the capture area; and
a porous insert inserted into the chamber and in contact with the buffer and/or the biological sample; wherein the porous insert defines a height of the gasketed area and limits free flow space in the gasketed area, wherein the substrate comprises a capture probe attached to the capture area, wherein the capture probe comprises a spatial barcode and a capture domain, and wherein the capture domain is hybridized directly or indirectly to a biological analyte from the biological sample or an intermediate agent indicative of the presence of the biological analyte.

41.-43. (canceled)

44. The composition of claim 40, wherein the porous insert comprises a hydrogel comprising of about 30% to about 50% poly(ethyleneglycol diacrylate).

45.-47. (canceled)

48. The composition of claim 40, wherein the porous insert comprises a plurality of pores, each pore having a diameter in a range from about 10 nm to about 100 μm; or wherein the porous insert has a contact angle between about 0° and about 80°; or wherein the porous insert has a compressibility between about 1×10⁻⁵ m²/N and about 1×10⁻⁸ m²/N.

49.-50. (canceled)

51. The method of claim 1, wherein the porous insert comprises a plurality of pores, each pore having a diameter in a range from about 10 nm to about 100 μm; or wherein the porous insert has a contact angle between about 0° and about 80°; or wherein the porous insert has a compressibility between about 1×10⁻⁵ m²/N and about 1×10⁻⁸ m²/N.

52. The method of claim 16, wherein the porous insert comprises a plurality of pores, each pore having a diameter in a range from about 10 nm to about 100 μm; or wherein the porous insert has a contact angle between about 0° and about 80°; or wherein the porous insert has a compressibility between about 1×10⁻⁵ m²/N and about 1×10⁻⁸ m²/N.

53. A kit comprising the composition of claim 40.
